(19) United States
(12) Patent Application Publication
(10) Pub. No.: US 2018/0259516 A1
(43) Pub. Date: Sep. 13, 2018

(54) MEANS AND METHODS FOR DIAGNOSING AND TREATING INFLAMMATORY DISORDERS

(71) Applicant: Westfälische Wilhelms-Universität Münster, Münster (DE)

(72) Inventors: Dirk Foell, Münster (DE); Christoph Kessel, Münster (DE)

(21) Appl. No.: 15/571,374

(22) PCT Filed: May 4, 2016
(86) PCT No.: PCT/IB2016/052538

§ 371 Date: Nov. 2, 2017

(30) Foreign Application Priority Data
May 4, 2015 (LU) 92707
May 12, 2015 (EP) 15167297.9

(51) Int. Cl. G01N 33/564; C07K 16/24; G01N 33/68

(52) U.S. Cl. CPC G01N 33/564; C07K 16/24; G01N 33/6851; A61P 37/06; G01N 2800/24; G01N 2800/7095; G01N 2800/102

(57) ABSTRACT

The present invention relates to a method of diagnosing or treating a S100A12:TLR4/MD2/CD14-mediated inflammatory disorder in a subject. In particular, the present invention is related to a compound having binding affinity to hexameric S100A12 complex and its use in diagnosis and therapy.

The present application provides for an in vitro method of diagnosing the risk of occurrence or the presence of a S100A12:TLR4/MD2/CD14-mediated inflammatory disorder in a subject, comprising determining the amount of hexameric S100A12 complex in a biological sample obtained from said subject, and comparing the amount of the hexameric S100A12 with a control sample. Moreover, the present invention relates to an immunoglobulin having a binding specificity to hexameric S100A12 complex but no binding affinity to tetrameric or dimeric S100A12 complex or monomeric S100A12. This immunoglobulin can be used in a method of diagnosing a S100A12:TLR4/MD2/CD14-mediated inflammatory disorder or in a method of treating a S100A12:TLR4/MD2/CD14-mediated inflammatory disorder. Moreover, this immunoglobulin can be used for the preparation of a diagnostic composition or may be comprised in a pharmaceutical composition. Also provided herein is a method for the treatment of a subject suffering from a S100A12:TLR4/MD2/CD14-mediated inflammatory disorder comprising administering a therapeutically effective amount of a compound having a binding specificity to hexameric S100A12 complex but no binding affinity to tetrameric or dimeric S100A12 complex or monomeric S100A12 to a subject in need thereof. The present invention also provides for a method of monitoring or evaluating the progression of an S100A12:TLR4/MD2/CD14-mediated inflammatory disorder

The so-called “Pattern Recognition” receptors (PRRs) are a central barrier of the innate immune system in the defense of the body against invading pathogens. PRRs recognize recurring molecular patterns of viruses and bacteria, such as components of the cell membrane; flagella or nucleic adds. One of the most prominent PRRs is the Toll-like receptor 4 (TLR4). TLR4 is primarily expressed on the surface of myeloid cells (monocytes, granulocytes, and dendritic cells). As a cell membrane component of gram-negative bacteria lipopolysaccharide (LPS) is the primary ligand for TLR4. An optimal TLR4 activation by LPS requires the formation of a signaling complex of TLR4 together with MD2 and CD14. However, TLR4-dependent signaling is not exclusively done by LPS, but also by other endogenous ligands. These are so-called “Damage Associated Molecular Pattern” (DAMP) proteins (also called alarmine), which are either passively released in the course of uncontrolled cell death (necrosis) or actively secreted due to cell stress. Their function can be seen in the initiation of a controlled inflammatory response in order to the remove necrotic cells (Piccinini A. M. & Midwood K. S. (2010), Mediators of inflammation 2010).

Also proteins that belong to the S100 (Calgranulin)-family such as S100A8/S100A9 and S100A12 are considered as alarmines and theft constitutive expression is mainly restricted to myeloid phagocytic cells (Foell, D. et al. (2007), Journal of Leukocyte Biology 81(1):28-37). These EF-hand calcium-modulated proteins are known as to be being associated with many diseases, including acute and chronic inflammatory disorder, neurological disorders and cancer. In the last years there has been an increasing interest in the function of S100A12, also known as Calgranulin C, MRP6, or EN-RAGE, which is predominantly secreted by neutrophil granulocytes, and its role as a systemic indicator of autoinflammatory diseases such as juvenile idiopathic arthritis (JIA) and other inflammatory conditions has been outlined (Vogl, T. et al, (1999), The Journal of Biological Chemistry 274(36): 25291-25296; Meijer, B. et al. (2012), International Journal of Inflammation Vol. 2012, Article ID 907078).

The 92 amino acid residue protein 51000A12 can oligomerize to higher oligomeric states depending on metal-ions like Ca²⁺, Zn²⁺, Na⁺ or Cu²⁺, wherein S100A12 oligomers such as dimers (dimS100A12), tetramers (tetS100A12) and hexamers (hexS100A12) exist in equilibrium depending on the local metal-ion concentration. S100A12 hexamers are said to be formed in the presence of (only) Ca²⁺ or Zn²⁺, or Zn²⁺/Ca²⁺ ions (Moroz, O. V. et al. (2009), BMC Biochemistry 10:11; Moroz, O. V. et al (2009), Journal of Molecular Biology 391(3):536-551; Koch, M. et al. (2008), Journal of Molecular Biology 378(4):933-942); Baudier, J. et al. (1986), The Journal of Biological Chemistry 261(18):8192-8203; Dell'Angelica, E. C. (1994), The Journal of Biological Chemistry 269(46):28929-28936).

In the clinic, S100A12 and S100A8/A9 are considered as excellent inflammatory biomarkers, overexpressed in a variety of inflammatory diseases. Because of their good correlation with inflammatory processes, they are suitable for monitoring the process of inflammation, in particular to trace results of therapy or to predict recurrence of inflammation in chronic inflammatory diseases (Kessel, C. et al. (2013), Clin. Immunol. 147(3):229-241). Due to the prevalence of S100A12 in autoinflammatory inflammation pattern the direct involvement of the protein in inflammatory processes via binding to the receptor of Advanced Glycation Endproducts (RAGE) was postulated (Hofmann, M. A. et al. (1999), Cell 97(7):889-901). However, recently it could be demonstrated that a pro-inflammatory stimulation of human monocytes with S100A12 protein is rather mediated via TLR4- instead of RAGE-activation, establishing the role of S100A12 as a mediator facilitating inflammatory monocyte activation in a cross-talk with activated granulocytes (FoeII, D. et al. (2013) American Journal of Respiratory and Critical Care Medicine 187(12):1324-1334).

Although S100A12 oligomerization and changes in protein conformation in dependence on metal ions were previously reported in the art and the physiological inflammatory potential of S100A12 protein in the interaction with TLR4 and in TLR4/MD2/CD14 signaling had been described, the prior art had failed up to now to demonstrate that higher S100A12 oligomeric states play a significant physiological role in TLR4/MD2/CD14-mediated signaling and inflammatory disorders. In fact, up to now there is no hint on which of the oligomeric S100A12 complexes is the deciding, physiologically active complex for the interaction with and the activation of TLR4/MD2/CD14-expressing monocytes and TLR4/MD2/CD14-mediated signaling in vivo.

With regard to the state of the art, there is a need to identify and characterize biomarkers of inflammation which are involved in the pathophysiology of inflammatory disorders such as autoinflammatory diseases, in order to better understand the underlying mechanism. In particular there is a demand for specific biomarkers of TLR4/MD2/CD14-mediated inflammatory disorders and for compounds suitable to diagnose said biomarkers, which would be of great interest for designing improved diagnostics. Additionally, diagnosis of new biomarkers involved in the pathophysiology of inflammatory disorders would be of great relevance for understanding the development of such disorders and to monitor clinically relevant inflammatory disorders on a molecular level. Moreover, there is a need in the art for new inhibitors of inflammatory disorders in drug discovery and development, particularly of TLR4/MD2/CD14-mediated inflammatory disorders, wherein such inhibitors would be extremely useful to improve the situation of patients suffering from chronic or acute autoinflammatory diseases and to adjust the treatment regimen of said patients. Such compounds could be utilized to design new medicaments and would allow for precisely targeted therapeutic approaches. In sum, there is a need to provide new, alternative means and methods that may help to diagnoses inflammatory disorders and to treat patients suffering from such inflammatory disorders, particularly TLR4/MD2/CD14-mediated disorders.

The technical problem underlying the present application is thus to comply with this need. The technical problem is solved by providing the embodiments reflected in the claims, described in the description and illustrated in the examples and figures that follow.

The present invention is, at least partly, based on the surprising finding that hexameric S100A12 complexes could be identified as being responsible for inflammatory signaling through TLR4. In vitro binding assays as well as cell stimulation experiments using chemically cross-linked HPLC-separated S100A12-oligomers revealed the S100A12 hexamers as the paramount TLR4-targeting inflammatory active complex. Stimulation could be abrogated by interfering with TLR4-binding and, in particular, by blocking access to CD14. More precisely, in receptor-interaction studies it could be shown that among artificially formed recombinant S100A12 di-, tetra- and hexamers (FIG. 1), the S100A12 hexamers are the predominant complex binding TLR4 and inducing IL-8 secretion by TLR4/MD2/CD14 expressing HEK-TCM cells, TNFα secretion by primary human monocytes and IL-1b, IL-6, IL-8 and TNFα expression in primary human monocytes, while a dimer and a tetramer were essentially not able to effect such a reaction on TLR4-expressing cells (FIG. 4, FIG. 7E, FIG. 8C). Moreover, it could be demonstrated by the present inventors that the formation of a hetero-tetramer TLR4/MD2-complex as well as CD14 seems to be generally required for optimal signal induction by hexS100A12, since CD14-blocking in TLR4/MD2/CD14-expressing human 293 cells (HEK-TCM) led to a reduced stimulation of the cells by the hexameric complexes (FIG. 5). Thus, the data of the present invention indicate that oligomerization of S100A12 to hexamers plays the deciding role in TLR4/MD2/CD14-mediated signaling. This was unforeseeable, as hexS100A12 was not reported so far as to be involved in TLR4/MD2/CD14-activation.

Moreover, the inventors performed extensive chemical crosslinking studies to assess S100A12-oligomerisation of both recombinant as well as native protein in autoinflammatory patients' sera as well as inside granulocytes. Only combined presence of Ca²⁺ and Zn²⁺ concentrations in extracellular ionic-strength could induce S100A12 hexamer-formation. Intracellular Ca²⁺/Zn²⁺-levels could only induce oligomerization up to the tetrameric complex. Notably, the inventors of the present invention were able to form Ca²⁺/Zn²⁺ S100A12 hexamers which were prepared under physiological extracellular metal ionic concentrations (25 mM Ca²⁺ and 1 mM Zn²⁺) and leading to a shift of the resulting protein complexes to the hexameric form (FIG. 2). The inventors were, despite the availability of technical means and established techniques for the artificial generation of di-, tetra and hexamers and by testing a wide range of concentrations not able to form hexamers in the presence of either Ca²⁺ or Zn²⁺ alone as described in the state of the art (Goyette, J. et al. (2009), Journal of Immunology 183(1):593-603; Xie, J. et al (2007), The Journal of Biological Chemistry 282(6):4218-4231). Further, S100A12 hexamers did likewise not form in presence of Ca²⁺ and Zn²⁺ concentrations within the physiological intracellular range. Hexamerization of S100A12 was only observed in presence of physiological extracellular concentrations of both Ca²⁺ and Zn²⁺. Thus, the present invention provides the first evidence that naturally-occurring (i.e. under physiological conditions) S100A12 hexamers are Ca²⁺/Zn²⁺ hexamers that are formed in the presence of physiological concentrations of Ca²⁺ and Zn²⁺. This was unexpected since the formation of only Ca²⁺ and Zn²⁺-dependent S100A12 hexamers had been described in the art, wherein Zn²⁺-dependent hexamers seemed to be the more relevant complex form in vivo (Goyette, J. et al. (2009), Journal of Immunology 183(1):593-603).

Correspondingly, the inventors showed that S100A12 hexamers are predominantly present in sera from patients suffering from an autoinflammatory disease. In fact, S100A12 hexamers could be detected in serum of these patients while this particular protein complex could not be found inside granulocytes, despite the overall high S100A12 concentrations naturally found inside these cells (FIG. 6). Hexameric S100A12 complexes from patient sera could be further applied head-to-head with an artificially formed S100A12 hexamer in in vitro binding assays as well as cell stimulation experiments, confirming the results obtained for artificially formed hexameric S100A12 complexes (FIG. 7, 8). Accordingly, the present invention discloses that under physiological conditions Ca²⁺/Zn²⁺ S100A12 hexamers indeed reflect the deciding, naturally-occurring form of S100A12 protein that plays a role in the interaction with TLR-4. S100A12mers were obtained from such sera by immunoprecipitation and were subsequently subject to gel electrophoresis in order to determine their degree of di-, tetra- or hexamerization. Given the fact that artificially formed Ca²⁺/Zn²⁺ hexS100A12 as well as Ca²⁺/Zn²⁺ hexS100A12 isolated from patient's serum effect a response in vitro (FIG. 4, 5, 7, 8), it is apparent that the hexameric form of S100A12 protein plays the decisive physiological role in the interaction with TLR4/MD2/CD14-complex also under in vivo conditions. Consequently, the specific detection and quantification of hexameric S100A12 in patient's material has practical implications in the diagnosis of inflammatory states. Moreover, these findings suggest the treatment of patients suffering from a S100A12-mediated inflammatory disorder by blocking the interaction of hexameric S100A12 complex to TLR4/MD2/CD14 complex. In sum, the means and methods of the present invention allow for detecting, diagnosing, monitoring, treating etc. a S100A12:TLR4/MD2/CD14-mediated inflammatory disorder in a subject.

Accordingly, on a general basis, the present invention relates to methods and uses of diagnosing and methods and uses of treating a S100A12-mediated disorder, i.e. a disorder, condition, or disease state characterized by TLR4/MD2/CD14-activation and inflammation. Such a S100A12-mediated disorder includes excessive TLR4/MD2/CD14-signaling induced by extracellular hexameric S100A12 complexes formed under physiological conditions. In a specific aspect, the TLR4/MD2/CD14-signaling is a level of TLR4/MD2/CD14-signaling in a cell or tissue suspected of being diseased that exceeds the level of TLR4 MD2/CD14-signaling in a similar non-diseased cell or tissue. It is envisaged that compounds for treating a S100A12-mediated disorder as disclosed herein allow for blocking or reducing the TLR4/MD2/CD14-signaling activity and that the formation of a complex between hexameric S100A12 and a TLR4 receptor is reduced, including prevented. It is further envisaged that compounds for diagnosing a S100A12-mediated disorder as disclosed herein allow for a precise detection of extracellular hexameric S100A12 complexes formed under physiological extracellular Ca²⁺/Zn²⁺-concentrations.

Thus, in a first aspect, the present invention relates to an in vitro method of diagnosing the risk of occurrence or the presence of a S100A12:TLR4/MD2/CD14-mediated inflammatory disorder in a subject, comprising:
(a) determining the amount of hexameric S100A12 complex in a biological sample obtained from said subject, and
(b) comparing the amount of hexameric S100A12 complex determined in (a) with a control sample.

The term “diagnosing the risk of occurrence” when used herein refers to evaluating whether a subject may be of a risk to develop a S100A12:TLR4/MD2/CD14-mediated inflammatory disorder. The term “diagnosing the presence” when used herein refers to evaluating whether a subject already suffers from a S100A12:TLR4/MD2/CD14-mediated inflammatory disorder. In this regard, an increased amount of hexameric S100A12 complex as compared to a control sample is indicative of an elevated risk of occurrence or the presence of said S100A12:TLR4/MD2/CD14-mediated inflammatory disorder. In some embodiments the method of diagnosing is a method of an early stage diagnosis. The term “early stage” as used herein encompasses, but is not limited to medical conditions in which the afflicted subject, e.g. the afflicted human subject, has little to no external sign of an S100A12:TLR4/MD2/CD14-mediated inflammatory disorders and the overall health situation is apparently preserved.

In the context of the present invention, the S100A12:TLR4/MD2/CD14-mediated inflammatory disorder is typically characterized by an increased amount of extracellular hexameric S100A12 complex in said subject. The term “increased amount” when used herein with respect to hexS100A12 means that the measured level or value of hexameric S100A12 complex in a biological sample from a subject is significantly higher as compared to the measured level of hexameric S100A12 complex in a control sample. The term “control sample” when used herein can be equally substituted with the term “reference sample”, wherein a control or reference sample comprises a biological sample obtained from a subject known not to be at the risk of occurrence or not having a S100A12:TLR4/MD2/CD14-mediated inflammatory disorder. In some embodiments a respective reference sample refers to a sample from a subject that is age-matched. In some embodiments such a reference sample refers to a sample from the same subject taken at a previous point of time. In a method as disclosed herein the amount of hexameric S100A12 complex as determined in a biological sample obtained from a subject may be compared to such a reference sample. In some embodiments the amount of hexameric S100A12 complex as determined in a biological sample obtained from a subject is compared to a threshold value. Such a threshold value may in some embodiments be a predetermined threshold value. In some embodiments the threshold value is based on the amount of hexameric S100A12 complex determined in a control sample. Generally, a respective control sample may have any condition that varies from the sample used in the main measurement.

The term “sample” when used herein relates to a material or mixture of materials, typically but not necessarily in liquid form, containing one or more analytes of interest. Preferably, the sample of the present invention is a biological sample. The term “biological sample” as used herein refers to a sample obtained from a subject, wherein the sample may be any biological tissue or fluid sample. Frequently, the sample will be a “clinical sample” which is a sample derived from a patient. Preferably, the biological sample of the present invention is a serum sample, a plasma sample, an urine sample, a feces sample, a saliva sample, a tear fluid sample, or a tissue extract sample. Further samples envisaged are sputum, cerebrospinal fluid, fine needle biopsy samples, peritoneal fluid, and pleural fluid, but the invention is not limited thereto. In the context of the present invention, said biological sample preferably relates to extracellular material obtained from said subject, i.e. extracellular body fluids. Biological samples may also include sections of tissues such as frozen sections taken for histological purposes. Such samples include, for example, whole blood, serum, etc. Preferably, a sample is a sample that includes extracellular material or liquid. Biological samples can be analyzed directly or they may be subject to some preparation prior to use in methods or assays of this invention. Such preparation can include, but is not limited to, suspension/dilution of the sample in water or an appropriate buffer or removal of cellular debris, e.g. by centrifugation, or selection of particular fractions of the sample before analysis.

The term “subject” as used herein, also addressed as an individual, refers to a mammal. The mammal may be any one of mouse, rat, guineas pig, rabbit, cat, dog, monkey, horse, or human. Accordingly, the mammal of the present invention may be a human or a non-human mammal. Thus, the methods, uses and compositions described in this document are generally applicable to both human and non-human mammals. As explained elsewhere herein, a sample may be analyzed that has been obtained from said subject, which is typically a living organism. Where the subject is a living human who may receive treatment or diagnosis for a disease or condition as described herein, it is also addressed as a “patient”.

In some embodiments the subject of the present invention is of a risk to develop an inflammatory disorder. In some embodiments the subject of the present invention suffers from an inflammatory disorder. The term “inflammatory disorder” as used herein comprises acute or chronic inflammatory diseases such as autoinflammatory diseases or other inflammatory conditions. The inflammatory disorder according to the present invention is generally a S100A12:TLR4/MD2/CD14-mediated inflammatory disorder characterized by an increased amount of extracellular hexameric S100A12 complex in said subject. Preferably, the extracellular hexameric S100A12 complex is a Ca²⁺/Zn²⁺-dependent hexameric S100A12 complex, wherein said complex is formed under physiological Ca²⁺/Zn²⁺-concentrations. The particular physiological role of hexameric S100A12 in TLR4/MD2/CD14-mediated inflammatory disorders has been underlined by experimental studies showing the stimulation of TLR4/MD2/CD14-expressing human 293 cells (HEK-TCM) with recombinant hexS100A12 as well as hexS100A12 complexes isolated from the serum of a patient, inducing expression and secretion of inflammatory cytokines such as TNFα, IL-8, IL-1b, and IL-6 by primary human monocytes, while a dimer and a tetramer were essentially not able to effect such a reaction on TLR4-expressing cells (FIGS. 4, 5, 7 and 8). Thus, in the context of the present invention, the TLR4/MD2/CD14-mediated inflammatory disorder is further characterized by an increased expression and/or accumulation of TNFα, IL-8, IL-1b, and IL-6. Typical examples of inflammatory disorder in the context of the present invention are e.g. acute and chronic inflammatory diseases selected from the group consisting of inflammatory bowel disease, juvenile idiopathic arthritis (JIA), systemic juvenile idiopathic arthritis (sJIA), rheumatoid and psoriatic arthritis, seronegative arthritis, or local and systemic inflections, vasculitides, cancer, kidney disease malfunctions, lung injury or pulmonary diseases, allergies, cardiovascular diseases, familial Mediterranean fever (FMF), or pyoderma gangrenosum and acne (PAPA), just to name some.

Although in principle all oligomeric states of S100A12 protein are present in a subject in vivo, the data of the present invention disclose that oligomerization of S100A12 to hexamers plays a deciding role in inflammatory disorders and TLR4/MD2/CD14-mediated signaling. The term “hexameric S100A12”, “hexS100A12” or “S100A12 hexamer” when used herein refers to a homomultimeric S100A12 protein formation consisting of six monomeric S100A12 subunits. In the context of the present invention, the hexameric S100A12 complex is preferably an extracellular Ca²⁺/Zn²⁺-dependent hexameric complex which is formed as follows. The binding of metal ions influences the conformation and oligomerization of S100A12. The Ca²⁺ binding induces a C-terminal conformational change that exposes a hydrophobic region of the surface over which two monomers in antiparallel orientation form a dimer. According to the current state of knowledge, this non-covalently linked dimer (21 kDa) is the smallest physiologically relevant S100A12 complex form. The Zn²⁺ binding of dimS100A12 induces their organization in homo-tetramers (44 kDa). This increases the Ca²⁺ affinity of the participating S100A12 monomers. Further Ca²⁺-binding subsequently induces hexamer formation (63 kDa). Accordingly, S100A12 hexamers much differ in the architecture of their quaternary structure from tetrameric protein (FIG. 1). While only combined presence of Ca²⁺ and Zn²⁺ concentrations in extracellular ionic-strength could induce S100A12 hexamer formation, intracellular Ca²⁺/Zn²⁺-levels could only induce oligomerization up to the tetrameric complex (FIG. 2). Accordingly, the extracellular Ca²⁺/Zn²⁺-dependent hexameric complex of the present invention are preferably complexes formed under physiological extracellular Ca²⁺/Zn²⁺-concentrations. The term “physiological extracellular Ca²⁺/Zn²⁺-concentration refers to any Ca²⁺/Zn²⁺-concentration that allows for an oligimerization of S100A12 tetramers to S100A12 hexamers. Preferably, the physiological extracellular Ca²⁺-concentration is at least 25 mM or higher and the physiological extracellular Zn²⁺-concentration is at least 1 mM or higher.

The term “determine” or “determining”, as well as “detect” or “detecting” when used in the context of a biomarker, preferably in the context of S100A12, refers to any measuring method that can be used to identify all forms of the S100A12 protein, i.e. hexameric, tetrameric, dimeric S100A12 complexes and monomeric S100A12 protein expressed by a cell. When used herein in combination with the words “level”, “amount” or “value”, the words “determine” or “determining” or “detect” or “detecting” are understood to refer to a quantitative as well as a qualitative level. Accordingly, the present invention also provides for a method of quantifying hexameric S100A12 complex in a biological sample.

“Determining” or “quantifying” the amount of hexameric S100A12 complex or any other form of S100A12 protein in a biological sample can be carried out by way of any suitable technique available and known to those skilled in the art. In some embodiments determining the amount of hexameric S100A12 complex in a biological sample comprises the use of mass spectrometry. When applying mass spectrometry, the type of the different S100A12 oligomers is chemically identified and analyzed in a sample by measuring the mass-to-charge ratio and abundance of gas-phase ions. Mass spectrometry works by ionizing chemical compounds to generate charged molecules or molecule fragments and measuring their mass-to-charge ratios. In this regard, spectra are used to determine the elemental or isotopic signature of a sample, the masses of particles and of molecules, and to elucidate the chemical structures of molecules. In some embodiments determining the amount of hexameric S100A12 complex in a biological sample comprises the use of aptamer-target-binding technology. When applying aptamer-target-binding technology, S100A12 complexes are identified by a class of small nucleic acid ligands (aptamers). In some embodiments the aptamers are composed of RNA having high specificity and affinity for their targets. In some embodiments the aptamers are composed of single-stranded DNA oligonucleotides having high specificity and affinity for their targets. Similar to antibodies, aptamers interact with their targets by recognizing a specific three-dimensional structure and are thus termed “chemical antibodies.” In contrast to protein antibodies, aptamers offer unique chemical and biological characteristics based on their oligonucleotide properties. In other embodiments “determining” or “quantifying” the amount of hexameric S100A12 complex in a sample comprises the use of an immunoglobulin having binding specificity to hexameric S100A12 complex but no binding specificity to tetrameric or dimeric S100A12 complex or monomeric S100A12. Examples of suitable immunoassay techniques in this regard are radiolabel assays such as a Radioimmunoassay (RIA) or enzyme-immunoassay such as an Enzyme Linked Immunosorbent Assay (ELISA), Luminex®-assays, precipitation (particularly immunoprecipitation), a sandwich enzyme immune test, an electro-chemiluminescence sandwich immunoassay (ECLIA), a dissociation-enhanced lanthanide fluoro immuno assay (DELFIA), a scintillation proximity assay (SPA), turbidimetry, nephelometry, latex-enhanced turbidimetry or nephelometry, or a solid phase immune test. Further methods known in the art (such as gel electrophoresis, 2D gel electrophoresis, SDS polyacrylamid gel electrophoresis (SDS-PAGE), and Western Blotting, can be used alone or in combination with labelling or other detection methods as described herein.

Within the context of the present invention, an ELISA or RIA test can be competitive for measuring the amount of hexS100A12, i.e. the amount of antigen. For example, an enzyme labelled antigen is mixed with a test sample containing antigen, which competes for a limited amount of immunoglobulin. The reacted (bound) antigen is then separated from the free material, and its enzyme activity is estimated by addition of substrate. An alternative method for antigen measurement is the double immunoglobulin sandwich technique. In this modification a solid phase is coated with specific immunoglobulin. This is then reacted with the sample from the subject that contains the antigen. Then enzyme labelled specific immunoglobulin is added, followed by the enzyme substrate. The ‘antigen’ in the test sample is thereby ‘captured’ and immobilized on to the sensitized solid phase where it can itself then immobilize the enzyme labelled immunoglobulin. This technique is analogous to the immunoradiometric assays. In an indirect ELISA method, an antigen is immobilized by passive adsorption on to the solid phase. A test serum may then be incubated with the solid phase and any immunoglobulin in the test serum forms a complex with the antigen on the solid phase. Similarly a solution of a proteinaceous binding molecule with immunoglobulin-like functions may be incubated with the solid phase to allow the formation of a complex between the antigen on the solid phase and the proteinaceous binding molecule. After washing to remove unreacted serum components an anti-immunoglobulin immunoglobulin anti-proteinaceous binding molecule immunoglobulin, linked to an enzyme is contacted with the solid phase and incubated. Where the second reagent is selected to be a proteinaceous binding molecule with immunoglobulin-like functions, a respective proteinaceous binding molecule that specifically binds to the proteinaceous binding molecule or the immunoglobulin directed against the antigen is used. A complex of the second proteinaceous binding molecule or immunoglobulin and the first proteinaceous binding molecule or immunoglobulin, bound to the antigen, is formed. Washing again removes unreacted material. In the case of RIA radioactivity signals are being detected. In the case of ELISA the enzyme substrate is added. Its colour change will be a measure of the amount of the immobilized complex involving the antigen, which is proportional to the antibody level in the test sample.

Luminex®-assays in the context of the present invention are designed to simultaneously measure multiple protein targets, i.e. different oligomeric forms of S100A12 complex in a single sample. In some embodiment the Luminex®-assay may be designed in a polystyrene format. In some embodiment the Luminex®-assay may be designed in a magnetic bead format. In some embodiments the Luminex®-assay used to analyze the individual S100A12 targets is a singleplex-assay. In some embodiments the Luminex®-assay is a multiplex assays.

“Comparing the amount” of hexameric S100A12 complex from a sample of a subject with a control sample when used herein means that said sample can be compared to a single control sample or a plurality of control samples, such as a sample from a control subject, in any suitable manner. As an illustrative example, the level of hexS100A12 in a control sample can be characterized by an average (mean) value coupled with a standard deviation value, for example at a given time point. In some embodiments the level of hexS100A12 in a subject may be considered increased or decreased when it is one standard deviation or more higher or lower than the average value of the corresponding heterodimer/tetramer determined in one or more control samples. In some embodiments the determined level of hexS100A12 is regarded as increased or decreased where the obtained value is about 1.5 standard deviations higher or lower, including about two, about three, about four or more standard deviations higher or lower than the average value determined in a control sample. In some embodiments the determined amount of hexS100A12 is regarded as different where the obtained value is about 1.2 times or more higher or lower, including about 1.5 times, about two fold, about 2.5-fold, about three fold, about 3.5 fold, about 4-fold, about 5-fold or more higher or lower than the protein level determined in a control sample.

According to various aspects, the present invention provides for a compound having a binding specificity to hexameric S100A12 complex but no binding specificity to tetrameric or dimeric S100A12 complex or monomeric S100A12. Preferably, this compound is an immunoglobulin having binding specificity to hexameric S100A12 complex but no binding specificity to tetrameric or dimeric S100A12 complex or monomeric S100A12. Although immunoglobulins having binding affinity to S100A12 protein have been described in the art, up to now there are no immunoglobulins available which specifically distinguish between the different S100A12-oligomers and particularly which have binding specificity to the physiologically most relevant hexameric S100A12 complex. In fact, the anti-S100A12-immunoglobulins described by Goyette et al. (2009, Journal of Immunology 183(1):593-603) are polyclonal antibodies that predominantly react with the tetrameric and hexameric form of S100A12 and thus miss specificity to solely hexameric S100A12. Accordingly, the immunoglobulins of the present invention that specifically bind to hexS100A12 are of great importance for use in diagnostic and therapeutic applications, which makes this finding particularly relevant. Thus, in one aspect, it is envisaged that the immunoglobulin as described herein is for use in a method of diagnosis. Preferably the method of diagnosis is a method of diagnosing a S100A12:TLR4/MD2/CD14-mediated inflammatory disorder as defined elsewhere herein. In some embodiments the use of the immunoglobulin of the present invention in a method of diagnosis comprises any of the detection methods as described elsewhere herein. In some embodiments the use involves an imaging technique. In some embodiments the imaging technique comprises histological staining of tissues sections obtained from a subject. The term “imaging” when used herein refers to the optical visualization of low or high levels of hexS100A12 by using the immunoglobulin of the present invention which is covalently linked to a label and any of the imaging techniques as described herein. In some embodiments low or high levels of hexS100A12 are visualized at local site of inflammation under in vivo conditions. In some embodiments low or high levels of hexS100A12 are visualized under in vitro conditions.

In some embodiments the imaging technique according to the present invention is a molecular imaging technique. Molecular imaging offers unique molecular sensitivity for preclinical and clinical studies and is generally used to explore physiological processes in real-time in vivo and to diagnose or certain diseases due to molecular abnormalities by means of imaging techniques. Moreover, the method of molecular imaging may also be applicable in recurrence diagnosis. Molecular imaging techniques generally involve inference from the deflection of light emitted from e.g. a laser or infrared source to structure, texture, anatomic and chemical properties of material. In some embodiments the molecular imaging technique is a non-invasive molecular imaging technique. Accordingly, an immunoglobulin for use in a method of diagnosing comprising molecular imaging techniques has to be covalently linked to a label, preferably a radioactive label. The term “label” in general refers to a moiety that allows detection and/or imaging. The skilled artisan is aware of a variety of labels suitable for use in molecular imaging techniques. Two illustrative examples of a suitable radioactive label are ¹²⁴I and ⁸⁹Zr, which may be coupled to the immunoglobulin by means of a chelating moiety. In some embodiments ⁶⁸Ga may also be used as a radioactive label. Positron emission tomography (PET) imaging may then be used. Typically these radiopharmaceuticals are intravenously applied. A typical PET scanner that is used in the art can detect concentrations between 10⁻¹¹ M and 10⁻¹² M, which is sufficient for the detection of S100A12. PET can quantitatively image the distribution of a radiolabeled immunoglobulin within the organism of the subject. Further molecular imaging techniques that may be used include, but are not limited to molecular magnetic resonance imaging (MRI), bioluminescence, fluorescence, targeted ultrasound, and single photon emission computed tomography (SPECT). An overview on molecular imaging techniques has been given by Dzik-Jurasz (The British Journal of Radiology (2003) 76 S98-S109). In some embodiments the immunoglobulin may be coupled to a nanoparticle such as a nanocrystal.

Where desired, an immunoglobulin as defined herein may be used in a hybrid imaging approach. For example, a PET/CT or a SPECT/CT camera is a commercially available combined system, which allows sequentially acquiring both anatomic and functional information that is accurately fused in a single examination. Integrated PET/magnetic resonance imaging allows for a correction for motion of organs or subjects. Magnetic resonance imaging also offers information about perfusion and blood flow, which may be desired in PET reconstruction and data analysis in the context of inflammation. Molecular imaging by means of an immunoglobulin or a proteinaceous binding partner may also be carried out in the form of photoacoustic tomography (PAT) or combined with PAT. PAT is based on the conversion from optical to ultrasonic energy. Currently PAT is carried out by irradiating the biological tissue to be imaged using a nanosecond-pulsed laser beam to engender thermal and acoustic impulse responses. Today, PAT is generally implemented as focused-scanning photoacoustic microscopy (PAM), photoacoustic computed tomography (PACT), and photoacoustic endoscopy (PAE).

In some embodiments the imaging technique according to the present invention is an optical imaging technique. Accordingly, the immunoglobulin of the present invention may further be covalently linked to optical imaging labels, e.g. dyes such as FITC, DiR or Alexa Fluor® 488, just to name some. These dyes are particularly useful in intraoperative applications. In some embodiments the immunoglobulin of the present invention for use in a method of diagnosis is covalently linked to fluorophores, e.g. polymethine dyes. Classical optical imaging techniques rely on the use of visible, ultraviolet, and infrared light in imaging. The skilled person is aware of different optical imaging systems applicable in combination with the compounds of the present invention. These systems are mainly divided into diffusive and ballistic imaging systems, which can all be used within the scope of the present invention.

As indicated herein, the immunoglobulin of the present invention can be coupled to an optically detectable label, a fluorophore, or a chromophore. Examples of suitable labels within the scope of the present invention include, but are not limited to, an organic molecule, an enzyme, a radioactive, fluorescent, and/or chromogenic moiety, a luminescent moiety, a hapten, digoxigenin, biotin, a metal complex, a metal and colloidal gold. Accordingly an excitable fluorescent dye, a radioactive amino acid, a fluorescent protein or an enzyme may for instance be used to detect e.g. the level of S100A12, in which the region required for binding to the TLR4 receptor is accessible. Examples of suitable fluorescent dyes include, but are not limited to, fluorescein isothiocyanate, 5,6-carboxymethyl fluorescein, Cascade Blue®, Oregon Green®, Texas red, nitrobenz-2-oxa-1,3-diazol-4-yl, coumarin, dansyl chloride, rhodamine, amino-methyl coumarin, DAPI, Eosin, Erythrosin, BODIPY®, pyrene, lissamine, xanthene, acridine, an oxazine, phycoerythrin, a Cy dye such as Cy3, Cy3.5, Cy5, Cy5PE, Cy5.5, Cy7, Cy7PE or Cy7APC, an Alexa dye such as Alexa 647, and NBD (Naphthol basic dye). Examples of suitable fluorescent protein include, but are not limited to, EGFP, emerald, EYFP, a phycobiliprotein such as phycoerythrin (PE) or allophycocyanin, Monomeric Red Fluorescent Protein (mRFP), mOrange, mPlum and mCherry. In some embodiments a reversibly photoswitchable fluorescent protein such as Dronpa, bsDronpa and Padron may be employed (Andresen, M., et al., Nature Biotechnology (2008) 26, 9, 1035). Regarding suitable enzymes, alkaline phosphatase, soybean peroxidase, or horseradish peroxidase may serve as a few illustrative examples. In some embodiments a method of detection may include electrophoresis, HPLC, flow cytometry, fluorescence correlation spectroscopy or a modified form of these techniques. Some or all of these steps may be part of an automated separation/detection system.

Further provided herein is the use of the immunoglobulin of the present invention for the preparation of a diagnostic composition for diagnosing a S100A12:TLR4/MD2/CD14-mediated inflammatory disorder in a subject, preferably suitable for use in any of the imaging techniques described elsewhere herein. Accordingly, the present invention provides for a diagnostic composition. The term “diagnostic composition” when used herein refers to a composition comprising any one of the immunoglobulins of the present invention and a diagnostically acceptable carrier, diluent or excipient, which can be applied for used in diagnosis. The carrier used in combination with the compound of the present invention is water-based and forms an aqueous solution. An oil-based carrier solution containing the compound of the present invention is an alternative to the aqueous carrier solution. Either aqueous or oil-based solutions further contain thickening agents to provide the composition with the viscosity of a liniment, cream, ointment, gel, or the like. Suitable thickening agents are well known to those skilled in the art. Alternative embodiments of the present invention can also use a solid carrier containing the diagnostic compound for use in diagnosis as disclosed elsewhere herein. This enables the alternative embodiment to be applied via a stick applicator, patch, or suppository. The solid carrier further contains thickening agents to provide the composition with the consistency of wax or paraffin.

Diagnostically acceptable excipients according to the present invention include, by the way of illustration and not limitation, diluent, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The diagnostic compositions can additionally include preservatives, solubilizers, stabilizers, wetting agents, emulsifiers, sweeteners, colorants, flavorings, buffers, coating agents, or antioxidants. Suitable diagnostic carriers are described in Remington's Pharmaceutical Sciences, Mack Publishing Company, a standard reference text in this field.

Further envisaged is the use of the diagnostic composition of the present invention as a diagnostic kit for the diagnosis of S100A12:TLR4/MD2/CD14-mediated inflammatory disorder characterized by an increased amount of extracellular hexameric S100A12 complex in said subject. Accordingly, the present invention further provides for a kit comprising the immunoglobulin as described elsewhere herein. In some embodiments the term “kit” when used herein refers to an assembly of useful compounds and other means like solid support plates or test stripes for detecting S100A12 hexameric complexes in a mammalian sample. A kit therefore may include the diagnostic composition of the present invention. Other components such as buffers, controls, and the like, known to those skilled in art, may be included in such test kits. The relative amounts of the various reagents can be varied, to provide for concentrations in solution of the reagents that substantially optimize the sensitivity of the assay. Particularly, the reagents can be provided as dry powders, usually lyophilized, which on dissolution will provide for a reagent solution having the appropriate concentrations for combining with a sample. The present kit may further include instructions for carrying out one or more methods of the present invention, including instructions for using standard and/or composition of the present invention that is included with the kit. In some embodiments the diagnostic kit comprises a monoclonal antibody exclusively binding to hexameric S100A12 complexes but not to tetrameric or dimeric S100A12 complexes or to monomeric S100A12. The antibodies used in said kit can be present in bound or soluble from.

The terms “specific” and “specificity” as used herein are understood to indicate that the compound of the present invention, in particular the immunoglobulin of the present invention, is directed against, binds to, or reacts with the hexameric S100A12 complex but not with the tetrameric or dimeric S100A12 complex or monomeric S100A12. Thus, “being directed to”, “binding to” or “reacting with” includes that the compound of the present invention specifically binds to a region of a S100A12 protein which is accessible in the hexameric state, but not accessible in the tetrameric, dimeric or monomeric state. The term “specifically” in this context means that the compound reacts with a corresponding region of hexS100A12 as applicable, or/and a portion thereof, but at least essentially not with another state of the S100A12 protein. The term “react essentially not” means that the compound does not have any particular affinity to other states of the S100A12 protein such as the monomeric, dimeric or tetrameric state, i.e. shows a cross-reactivity of less than about 30%, such as less than about 20%, less than about 10%, including less than about 9, 8, 7, 6 or 5%, when compared to the affinity to another state of the S100A12 protein. Whether the compound specifically reacts as defined herein above can easily be tested, inter alia, by comparing the reaction of a respective compound with monomeric, dimeric or tetrameric S100A12, as applicable. The term “specifically recognizing”, which can be used interchangeably with the terms “directed to” or “reacting with” means in the context of the present disclosure that a particular compound, generally an immunoglobulin is capable of specifically interacting with and/or binding to at least two, including at least three, such as at least four or even more amino acids of an epitope of the hexameric S100A12 protein complex as defined herein. Generally the immunoglobulin or compound as defined herein can thereby form a complex with the respective epitope of hexS100A12. Such binding may be exemplified by the specificity of a “lock-and-key-principle”. “Specific binding” can also be determined, for example, in accordance with Western blots, ELISA-, RIA-, ECL-, IRMA-tests, FACS, IHC and peptide scans.

In a preferred embodiment of the present invention, the respective compound having a binding specificity to hexameric S100A12 complex but no binding specificity to tetrameric or dimeric S100A12 complex or monomeric S100A12 is an antibody. The term “antibody” refers to a molecule in which the structure and/or function is/are based on the structure and/or function of an antibody, e.g. of a full-length or whole immunoglobulin molecule. The hexameric S100A12 complex bound by the immunoglobulin of the present invention is preferably a Ca²⁺/Zn²⁺-dependent hexameric S100A12 complex. According to the present invention, the immunoglobulin specifically inhibits the interaction of hexameric S100A12 complex to the TLR4/MD2/CD14 complex. In this regard the immunoglobulin blocks the TLR4/MD2/CD14-signaling. In a preferred embodiment the immunoglobulin significantly decreases the expression of TNFα, IL-8, IL-1b, and IL-6. As shown in FIG. 9A, the raised anti-S100A12 monoclonal antibody (mAB) clone 11G7A11 preferentially recognizes hexameric S100A12. Moreover, in primary human monocyte stimulation experiments using LPS-free hexameric S100A12, mAB 11G7A11 effectively neutralizes hexS100A12-induced TNFα-release by these cells (FIG. 9B).

An antibody construct of the present invention is hence capable of binding to its specific epitope or antigen. Furthermore, an antibody construct according to the invention comprises the minimum structural requirements of an antibody which allow for the epitope binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region). The antibodies on which the constructs according to the invention are based include for example monoclonal, recombinant, chimeric, deimmunized, humanized and human antibodies. Within the definition of “antibody” according to the invention are full-length or whole antibodies including camelid antibodies and other immunoglobulin antibodies generated by biotechnological or protein engineering methods or processes. These full-length antibodies may be for example monoclonal, recombinant, chimeric, deimmunized, humanized and human antibodies. Also within the definition of “antibody constructs” are fragments of full-length antibodies, such as VH, VHH, VL, (s)dAb, Fv, Fd, Fab, Fab′, F(ab′)2 or “r IgG” (“half antibody”). Antibodies according to the invention may also be modified fragments of antibodies, also called antibody variants, such as scFv, scFab, Fab2, Fab3, diabodies, single chain diabodies, “minibodies” exemplified by a structure which is as follows: (VH-VL-CH3)2, (scFv-CH3)2 or (scFv-CH3-scFv)2, and single domain antibodies such as nanobodies or single variable domain antibodies comprising merely one variable domain, which might be VHH, VH or VL, that specifically bind an antigen or epitope independently of other V regions or domains.

In some embodiments an antibody is an aptamer, including a Spiegelmer®, described in e.g. WO01/92655. An aptamer is typically a nucleic acid molecule that can be selected from a random nucleic acid pool based on its ability to bind a selected other molecule such as a peptide, a protein, a nucleic acid molecule a or a cell. Aptamers, including Spiegelmers, are able to bind molecules such as peptides, proteins and low molecular weight compounds. Spiegelmers® are composed of L-isomers of natural oligonucleotides. Aptamers are engineered through repeated rounds of in vitro selection or through the SELEX (systematic evolution of ligands by exponential enrichment) technology. The affinity of Spiegelmers to their target molecules often lies in the pico- to nanomolar range and is thus comparable to immunoglobulins. An aptamer may also be a peptide. A peptide aptamer consists of a short variable peptide domain, attached at both ends to a protein scaffold.

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins” or “Monoclonal antibodies” are substantially homogenous populations of immunoglobulins to a particular antigen. They may be obtained by any technique which provides for the production of immunoglobulin molecules by continuous cell lines in culture. Monoclonal immunoglobulins may be obtained by methods well known to those skilled in the art (see for example, Köhler et al., Nature (1975) 256, 495-497, and U.S. Pat. No. 4,376,110). An immunoglobulin or immunoglobulin fragment with specific binding affinity only for hexameric S100A12 complexes but no affinity for tetrameric or dimeric S100A12 complexes or monomeric S100A12 can be isolated, enriched, or purified from a prokaryotic or eukaryotic organism. Routine methods known to those skilled in the art enable production of both immunoglobulins and immunoglobulin fragments in both prokaryotic and eukaryotic organisms. In more detail, an immunoglobulin may be isolated by comparing its binding affinity to a protein of interest, i.e. hexameric S100A12 complex, with its binding affinity to other polypeptides, e.g. tetrameric or dimeric S100A12 complex or monomeric S100A12. Humanized forms of the antibodies may be generated using one of the procedures known in the art such as chimerization or CDR grafting.

In general, techniques for preparing monoclonal antibodies and hybridomas are well known in the art. Any animal such as a goat, a mouse or a rabbit that is known to produce antibodies can be immunized with the selected polypeptide, e.g. a human hexameric S100A12 complex. In a preferred embodiment, the immunoglobulin of the present invention is obtained by immunizing a rodent, preferably a rat, by immunization with a cross-linked hexameric S100A12 complex. These cross-linked hexameric S100A12 complexes are obtainable by complexation of recombinant S100A12 under physiological extracellular Ca²⁺/Zn²⁺-concentrations and cross-linking with Bis(Sulfosuccinimidyl) Substrate (BS³). More precisely, recombinant S100A12 is purified via anion-exchange chromatography followed by Ca²⁺-dependent hydrophobic interaction chromatography. The protein is dialyzed against HBS. To strongly shift the equilibrium of S100A12mers towards hexamers, the protein is incubated with 25 mM CaCl₂ and 1 mM ZnCl₂ for 30 minutes at RT. Resulting complexes are cross-linked by addition of BS³ and excessive BS³ is quenched. Crosslinked S100A12mers are separated via preparative gel filtration. Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection.

The polypeptide may be further modified or administered in an adjuvant in order to increase the peptide antigenicity. Methods of increasing the antigenicity of a polypeptide are well known in the art. Such procedures include coupling the antigen with a heterologous protein (such as globulin or b-galactosidase) or through the inclusion of an adjuvant during immunization. Typically, the immunized mammals are bled and the serum from each blood sample is assayed for particular antibodies using appropriate screening assays known to those skilled in the art.

For monoclonal immunoglobulins, lymphocytes, typically splenocytes, from the immunized animals are removed, fused with an immortal cell line, typically myeloma cells, such as SP2/0-Agl4 myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Any one of a number of methods well known in the art can be used to identify a hybridoma cell which produces an immunoglobulin with the desired characteristics. Typically the culture supernatants of the hybridoma cells are screened for immunoglobulins against the antigen. Suitable methods include, but are not limited to, screening the hybridomas with an ELISA assay, Western blot analysis, or radioimmunoassay (Lutz et al., Exp. Cell Res. 175, 109-124). Hybridomas prepared to produce anti-hexS100A12 immunoglobulins may for instance be screened by testing the hybridoma culture supernatant for secreted antibodies having the ability to bind to hexameric S100A12 complexes but no ability to bin tetrameric or dimeric S100A12 complexes or monomeric S100A12. To produce antibody homologs which are within the scope of the invention, including for example, anti-hexS100A12 antibody homologs, that are intact immunoglobulins, hybridoma cells that tested positive in such screening assays can be cultured in a nutrient medium under conditions and for a time sufficient to allow the hybridoma cells to secrete the monoclonal immunoglobulins into the culture medium. Tissue culture techniques and culture media suitable for hybridoma cells are well known in the art. The conditioned hybridoma culture supernatant may be collected and for instance the anti-hexS100A12 immunoglobulins optionally further purified by well-known methods. Alternatively, the desired immunoglobulins may be produced by injecting the hybridoma cells into the peritoneal cavity of an unimmunized mouse. The hybridoma cells proliferate in the peritoneal cavity, secreting the immunoglobulin which accumulates as ascites fluid. The immunoglobulin may be harvested by withdrawing the ascites fluid from the peritoneal cavity with a syringe.

Hybridomas secreting the desired immunoglobulins are cloned and the class and subclass are determined using procedures known in the art. For polyclonal immunoglobulins, immunoglobulin containing antisera is isolated from the immunized animal and is screened for the presence of immunoglobulins with the desired specificity using one of the above-described procedures. The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art.

A plurality of conventional display technologies is available to select an immunoglobulin or immunoglobulin fragment. Li et al. (Organic & Biomolecular Chemistry (2006), 4, 3420-3426) have for example demonstrated how a single-chain Fv fragment capable of forming a complex with a selected DNA adapter can be obtained using phage display. Display techniques for instance allow the generation of engineered immunoglobulins and ligands with high affinities for a selected target molecule. It is thus also possible to display an array of peptides or proteins that differ only slightly, typically by way of genetic engineering. Thereby it is possible to screen and subsequently evolve proteins or peptides in terms of properties of interaction and biophysical parameters. Iterative rounds of mutation and selection can be applied on an in vitro basis.

As described herein, the immunoglobulin of the present invention may be well suited to be used in a method of diagnosing a S100A12:TLR4/MD2/CD14-mediated inflammatory disorder in a subject. Moreover, the immunoglobulin having binding specificity to hexameric S100A12 complex but no binding specificity to tetrameric or dimeric S100A12 complex or monomeric S100A12 may further be used in therapy, in particular in the treatment of a condition, including an inflammatory disorders characterized by an increased amount of extracellular hexameric S100A12 complex in a subject. Accordingly, the present also relates to the immunoglobulin of the present invention for use in a method of treatment. Preferably said method of treatment is a method of treating a S100A12:TLR4/MD2/CD14-mediated inflammatory disorder in a subject. Also envisaged is the use of an immunoglobulin as described elsewhere herein for the treatment of a human subject suffering from a S100A12:TLR4/MD2/CD14-mediated inflammatory disorder. Accordingly, the present invention also relates to a method for the treatment of a subject suffering from a S100A12:TLR4/MD2/CD14-mediated inflammatory disorder, the method comprising administering a therapeutically effective amount of a compound having binding specificity to hexameric S100A12 complex but not binding specificity to tetrameric or dimeric S100A12 complex or monomeric S100A12 to a subject in need thereof. In the context of the present invention, the S100A12:TLR4/MD2/CD14-mediated inflammatory disorder is characterized by an increased amount of extracellular hexameric S100A12 complex in said subject. Preferably, the compound specifically inhibits the interaction of hexameric S100A12 complex to TLR4/MD2/CD14 complex, wherein said compound blocks the TLR4/MD2/CD14-signaling. IN some embodiments the compound significantly decreases the expression of TNFα, IL-8, IL-1b, and IL-6. Most preferred, the compound is an immunoglobulin as described elsewhere herein.

The term “treat”, “treating”, or “treatment” as used herein means to reduce, stabilize, or inhibit the progression of the symptoms associated with the inflammatory disorder. Said symptoms may be hallmarks of human pruritus such as epidermal hyperplasia, acanthosis, fibrosis, collagenosis, and/or an increased infiltration of lymphocyte like T-cells, mast cells, or eosinophiles into the dermis of said subject, just to name some. Those in need of treatment include those already with the disorder as well as those prone to having the disorder. Preferably, a treatment reduces, stabilizes, or inhibits progression of a symptom that is associated with the presence and/or progression of a disease or pathological condition. “Treat”, “treating”, or “treatment” refers to both therapeutic treatment and prophylactic or preventative measures, wherein the object is to prevent, slow down (lessen) or at least partially alleviate or abrogate an abnormal, including pathologic, condition in the organism. Those in need of treatment include those already with the disorder as well as those prone to having the disorder or those in whom the disorder is to be prevented (prophylaxis). The term “administering” relates to a method of incorporating a compound into cells or tissues of an organism.

The compounds for use in the treatment of a subject suffering from a S100A12:TLR4/MD2/CD14-mediated inflammatory disorder as described in the present invention are generally administered to the subject in a therapeutically effective amount. Said therapeutically effective amount is sufficient to inhibit or alleviate the symptoms of a S100A12:TLR4/MD2/CD14-mediated inflammatory disorder. By “therapeutic effect” or “therapeutically effective” is meant that the compound for use will elicit the biological or medical response of a tissue, system, animal or human that is being sought by the researcher, veterinarian, medical doctor or other clinician. The term “therapeutically effective” further refers to the inhibition of factors causing or contributing to the disease. The term “therapeutically effective amount” includes that the amount of the compound when administered is sufficient to significantly improve the progression of the disease being treated or to prevent development of said disease. The therapeutically effective amount will vary depending on the compound, the disease and its severity and on individual factor of the subject such. Therefore, the compound of the present invention will not in all cases turn out to be therapeutically effective, because the method disclosed herein cannot provide a 100% safe prediction whether or not a subject may be responsive to said compound, since individual factors are involved as well. It is to expect that age, body weight, general health, sex, diet, drug interaction and the like may have a general influence as to whether or not the compound for use in the treatment of a subject suffering from itch will be therapeutically effective. Preferably, the therapeutically effective amount of the compound used to treat a subject suffering from itch is between about 0.01 mg per kg body weight and about 1 g per kg body weight, such as about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 100, 200, 300, 400, 500, 600, 700, 800, or about 900 mg per kg body weight. Even more preferably, the therapeutically effective amount of the compound used to treat a subject suffering from itch is between about 0.01 mg per kg body weight and about 100 mg per kg body weight, such as between about 0.1 mg per kg body weight and about 10 mg per kg body weight. The therapeutic effective amount of the compound will vary with regard to the weight of active compound contained therein, depending on the species of subject to be treated.

The administration of the compounds for use in the treatment of a subject suffering from a S100A12:TLR4/MD2/CD14-mediated inflammatory disorder according to the present invention can be carried out by any method known in the art. In some embodiments, the administration is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes, or combinations thereof.

In the scope of the present invention, it is for example envisaged that the therapeutic effect is detected by way of surgical resection or biopsy of the affected skin or the effected tissue, which is subsequently analyzed by way of, for example immunological techniques. Alternatively, it is also envisaged that biomarkers in the skin of the patient are detected in order to diagnose whether or not the therapeutic approach is effective. Additionally or alternatively, it is also possible to evaluate the general appearance of the respective patient, which will also aid the skilled practitioner to evaluate whether the therapy is effective. Those skilled in the art are aware of numerous other ways which will enable a practitioner to observe a therapeutic effect of the compound for use in the treatment of itch as disclosed herein in the context of a method or use of the present invention.

While it is possible to administer the immunoglobulin of the present invention directly without any formulation, in another aspect of the present invention the compounds are preferably employed in the form of a pharmaceutical or veterinary formulation composition, comprising a pharmaceutically or veterinarily acceptable carrier, diluent or excipient and a compound of the present invention, preferably the immunoglobulin of the present invention. The carrier used in combination with the compound of the present invention is water-based and forms an aqueous solution. An oil-based carrier solution containing the compound of the present invention is an alternative to the aqueous carrier solution. Either aqueous or oil-based solutions further contain thickening agents to provide the composition with the viscosity of a liniment, cream, ointment, gel, or the like. Suitable thickening agents are well known to those skilled in the art. Alternative embodiments of the present invention can also use a solid carrier containing the compound for use in the treatment of a S100A12:TLR4/MD2/CD14-mediated inflammatory disorder as disclosed elsewhere herein. This enables the alternative embodiment to be applied via a stick applicator, patch, or suppository. The solid carrier further contains thickening agents to provide the composition with the consistency of wax or paraffin.

Alternatively, the compound for use in the treatment of a subject suffering from a S100A12:TLR4/MD2/CD14-mediated inflammatory disorder can be administered in a combination with another anti-inflammatory drug and/or a compound used to treat S100A12:TLR4/MD2/CD14-mediated inflammatory disorders. Many compounds are known to have anti-inflammatory effects and are therapeutically used to treat inflammatory conditions. These anti-inflammatory drugs and/or compounds used to treat a S100A12:TLR4/MD2/CD14-mediated inflammatory may be selected from the group consisting of an antihistamine, a glucocorticosteroid, a calcineurin inhibitor, a local anesthetic, serotonin-reuptake inhibitors (SSRI), menthol, camphor, neuroleptic, a topical antidepressant, a tetracyclic, a neurokinin-1 receptor antagonist, a mu-opioid receptor antagonists, a kappa opioid receptor antagonist, a protease inhibitor, a protease-activated receptor antagonist, a gastrin-realizing peptide, a gastrin-releasing peptide receptor antagonist, a brain-derived natriuretic peptide (BNP), a brain-derived natriuretic peptide (BNP) receptor antagonist, a dynorhin receptor antagonist, a cytokine, a cytokine antagonist, a quinolone, a chemokine receptor antagonist, and a botulinum toxin, just to name some. Said combination according to the present invention can be administered as a combined formulation or separate from each other. Moreover, the compounds for use in the treatment of a subject suffering from itch according to the present invention can be combined with ultraviolet radiation therapy. In some embodiments the ultraviolet radiation therapy is UVA radiation. In some embodiments the ultraviolet radiation therapy is UVB radiation. In some embodiments the ultraviolet radiation therapy is PUVA radiation.

Pharmaceutical excipients according to the present invention include, by the way of illustration and not limitation, diluent, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants.

The compositions according to the present invention are preferably formulated in a unit dosage form, each dosage containing about 1 to about 500 mg, more usually about 5 to about 300 mg, of the active ingredient. The term “unit dosage form” as used herein refers to physically discreet units suitable as unitary dosages for human subjects or other mammals, each unit containing a predetermined quantity of active material calculated to produce the desired therapeutic effect, in association with a suitable pharmaceutical carrier. As used herein, a “dosage” refers to an amount of therapeutic agent administered to a patient. As used herein, a “daily dosage” refers to the total amount of therapeutic agent administered to a patient in a day.

The compounds disclosed herein are also useful for treating inflammatory disorders in domestic animals such as cats, dogs, rabbits, guinea pigs, cows, sheeps, and horses. Thus, the invention also provides a veterinary formulation comprising a compound for use in the treatment of a subject suffering from itch as defined elsewhere herein together with a veterinary acceptable diluents or carrier. Such formulations include in particular ointments, pour-on formulations, spot-on formulations, dips, sprays, mousses, shampoos, collar, and powder formulations.

An immunoglobulin as disclosed in this document may also be used in preventing a condition associated with an inflammatory disorder of a subject. The term “preventing” refers to decreasing the probability that a subject contracts or develops an abnormal inflammatory condition. Accordingly, in some embodiments the immunoglobulin of the present invention may be used in preventing any of the S100A12:TLR4/MD2/CD14-mediated inflammatory disorders described elsewhere herein.

As reported in the state of the art, S100A12 seem to be a well suited and reliable biomarker for diagnosing and monitoring inflammatory disorders. The term “biomarker” is defined as a physical sign or laboratory measurement that occurs in association with a natural or pathological process, and that has putative diagnostic and/or prognostic utility. More precisely, the term “biomarker” may comprise a protein or a gene encoding a protein/peptide, which is expressed at a lower or higher level by a cell under different cellular conditions. In the present disclosure, said biomarker is preferably expressed and released by a subject under native and/or pathological conditions, such as inflammatory conditions. The biomarker described herein is usually expressed and released by an immune cell, in particular neutrophil granulocytes. Under physiological conditions, S100A12 forms oligomeric complexes, wherein hexameric S100A12 is the predominant extracellular form involved in TLR4/MD2/CD14-signaling. Accordingly, measuring the level of the hexameric complex of said biomarkers can be used for diagnosing and/or monitoring an acute or chronic S100A12:TLR4/MD2/CD14-mediated inflammatory disorder.

Thus, according to another aspect, also provided herein is a method of monitoring or evaluating the progression of an S100A12:TLR4/MD2/CD14-mediated inflammatory disorder in a patient, the method comprising:
(a) determining the amount of hexameric S100A12 complex in a biological sample of said patient, and
(b) comparing the amount of hexameric S100A12 complex determined in (a) to reference data obtained from said patient at an earlier date, wherein the result of the comparison of (b) provides an evaluation of the progression of the S100A12:TLR4/MD2/CD14-mediated inflammatory disorder.
   In this regard, a significantly increased amount of hexameric S100A12 complexes as compared to the reference data obtained from said patient at an earlier date indicates a progression of the S100A12:TLR4/MD2/CD14-mediated inflammatory disorder in said patient. On the other hand, no change or a significantly decreased amount of hexameric S100A12 complexes as compared to the reference data obtained from said patient at an earlier date indicates no progression or a regression of the S100A12:TLR4/MD2/CD14-mediated inflammatory disorder in said patient.

In the context of the present invention, the term “monitoring or evaluating the progression” refers to any procedure or method used in vitro or in vivo to assess whether or not a patient suffering from a S100A12:TLR4/MD2/CD14-mediated inflammatory disorder is responsive to treatment with a therapeutic compound. In this context, particularly S100A12:TLR4/MD2/CD14-mediated chronic autoinflammatory diseases such as juvenile idiopathic arthritis (JIA) or other inflammatory conditions, e.g. tumor associated inflammation, can be assessed using any of the methods of the present invention. In particular, a method of monitoring or evaluating the progression of a S100A12:TLR4/MD2/CD14-mediated inflammatory disorder relates to monitoring or evaluating the level of hexameric S100A12 complex in a subject prior, during and after therapy with a therapeutic compound. The term “therapeutic compound” as used herein refers to any compounds suitable to treat an inflammatory disease characterized by an increased amount of extracellular hexameric S100A12 complex in said subject, wherein said hexameric S100A12 complex is preferably a Ca²⁺/Zn²⁺-dependent hexameric S100A12 complex. Preferably, said therapeutic compound leads to a decreased expression and/or accumulation of TNFα, IL-8, IL-1b, and IL-6 in said patient by inhibiting the interaction of hexameric S100A12 complex to the TLR4/MD2/CD14 complex. Even more preferably, said therapeutic compound is an immunoglobulin as described elsewhere herein.

As described herein, a method of monitoring or evaluating the progression of S100A12:TLR4/MD2/CD14-mediated inflammatory disorder might be particularly useful when treating a patient suffering from an inflammatory disease or disorder associated with increased amount of extracellular hexameric S100A12 complex in said subject with any medicament for alleviating or healing said inflammatory disease. Accordingly, the method of monitoring as described herein particularly refers to in vivo monitoring the therapeutic efficacy of a drug used in the treatment of inflammatory disorders. Hence, conclusions can be drawn during and/or after the treatment of a subject with the medicament as to whether said medicament may improve symptoms of an inflammatory disease when comparing to the physical conditions before start of treatment. Moreover, such monitoring or evaluation may help an attending physician to obtain the appropriate information to set the appropriate therapy conditions for the treatment of said inflammatory disease.

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the methods and uses described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “consisting”, “consisting of” and “consisting essentially of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

As described herein, “preferred embodiment” means “preferred embodiment of the present invention”. Likewise, as described herein, “various embodiments” and “another embodiment” means “various embodiments of the present invention” and “another embodiment of the present invention”.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects according to the invention. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

Several documents are cited throughout the text of this disclosure. Each of the documents cited herein (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material. Nothing herein is to be construed as an admission that the invention is not entitled to antedate such disclosure by virtue of prior invention.

The following description includes information that may be useful in understanding the present invention. It is not an admission that any of the information provided herein is prior art or relevant to the presently claimed inventions, or that any publication specifically or implicitly referenced is prior art.

### BRIEF DESCRIPTION OF THE DRAWINGS

FIG. 1: S100A12-quaternary structures. There are shown representations of the potential surface S100A12-quaternary structures based on the respective crystallographic data and a schematic representation of the respective monomer-organization. Surface representation are colored according to their hydrophobicity (orange: hydrophobic; white: neutral; blue: hydrophilic). Structural data for dimS100A12: 2WCB.pdb; tetS100A12: 1ODB.pdb; hexS100A12: 1GQM.pdb

FIG. 2: Ca²⁺/Zn²⁺ induced S100A12-complexing. Recombinant S100A12 was incubated with the indicated concentrations of ions and the resulting complexes were cross-linked with BS3. The complexes were separated by gel electrophoresis and stained with Coomassie. Ca²⁺ (A) and Zn²⁺ (B) in the intracellular concentration induce at the maximum 2×S100A12. Only extracellular Zn²⁺-concentrations (>10 μM; B-D) induce the formation of 4×S100A12, 6×S100A12 is exclusively formed by simultaneous extracellular Ca²⁺-concentration (D).

FIG. 3: Separation of ion-induced S100A12-quaternary structures. (A) Ca²⁺/Zn²⁺-induced and subsequently BS³-cross-linked S100A12 complexes are separated from each other by gel filtration. S100A12 hexamers, tetramers and dimers are formed depending on the presence of Zn²⁺- and/or Ca²⁺-ions (B, C). (D) The isolated S100A12-homomultimers can be visualized by Coomassie staining after gel electrophoresis.

FIG. 4: TLR4 binding and signal induction by S100A12 complexes. (A) Gel-filtrated S100A12 oligomers were tested in ELISA for binding to immobilized TLR4/MD2-complex. (B) TLR4/MD2/CD14-expressing TOM HEK-cells were tested for 4 h with LPS, untreated recombinant S100A12 and the different S100A12-complexes on induction of reporter gene expression (IL-8). Likewise, (C) primary human monocytes were stimulated for 4 h with LPS and S100A12-homomultimers, and the resulting TNFα secretion was quantified by ELISA. In the same cells, IL-1b, IL-8, IL-6, and TNFα gene expression was examined (D),

FIG. 5: CD14 blocking. TLR4/MD2/CD14-expressing HEK-TCM cells were stimulated for 4 h with different concentrations of LPS and the different S100A12-complexes in the presence or absence of an anti-CD14 antibody (InvivoGen), and subsequently tested for induction of reporter gene expression (IL-8). CD14 blocking reduced LPS- as well as hexamer-induced IL-8 expression to background levels.

FIG. 6: Isolation of S100A12 complexes. Chemically cross-linked S100A12 complexes were isolated from both healthy control as well as autoinflammatory diseased patients' sera (A) or granulocytes (B, C) by anti-S100A12 monoclonal antibody pull down. (A) Sera of either healthy controls or patients with known high serum S100A12 levels were treated with BS³ and S100A12 was precipitated using anti-S100A12 coated magnetic beads. Samples were cooked and identical protein concentrations were subjected to SDS-PAGE and anti-S100A12 Western Blot. Hexameric S100A12 is clearly the dominating S100A12 complex from present in serum, whereas hexS100A12 in patients' sera is even more prominent, despite samples at identical protein concentrations were analyzed. (B, C) Similarly, primary human granulocytes were treated with a membrane permeable cross-linker and S100A12 complexes were isolated from cell lysates by antibody-mediated pull down, separated on SDS-PAGE and detected by anti-S100A12 Western Blot. Despite the naturally very high S100A12-concentration in granulocytes, no hexameric S100A12 could be detected in untreated cells (B; C, lane 1) or granulocytes challenged with stimuli (phorbol 12-myristate 13-acetate (PMA), H₂O₂) known to result in strong S100A12 mobilization (C; lanes 2, 3).

FIG. 7: Direct isolation of S100A12 complexes from patient serum.

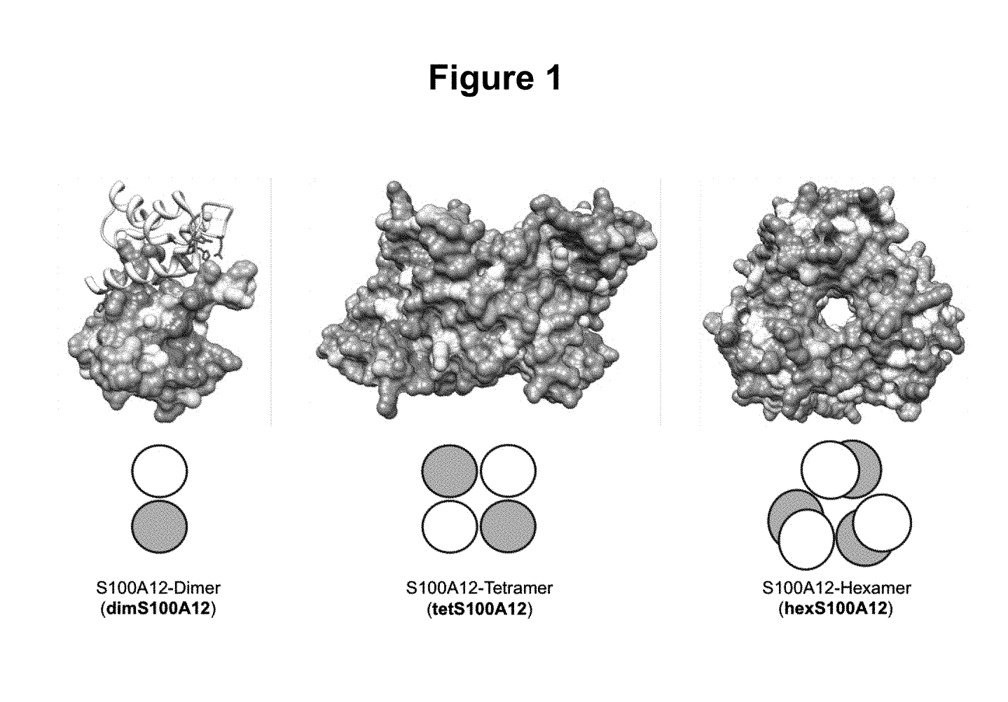

(A) Serum of an autoinflammatory diseased patient (PFAPA: Periodic fever, aphthous stomatitis, pharyngitis, adenitis syndrome) was treated with BS³ and S100A12 was precipitated using anti-S100A12 monoclonal antibody (3G1D5, produced in-house) coated magnetic beads. Beads were washed thoroughly to remove unspecifically bound proteins. Antibody-bound proteins were eluted by pH-shift. Subsequently, pH-neutralized eluted protein was separated by gel filtration (B).
(B, C) Gel filtrated protein was thoroughly fractionized and eluted fractions from identified protein peaks (1-7) were analyzed on SDS PAGE and subsequent Coomassie stain (C). In this analysis hexS100A12 appeared as the dominant band, followed by bands resembling multimers of hexS100A12 and a weak tetraS100A12 band. DimS100A12 cannot be detected in serum.
(D) Eluted protein fractions were pooled and protein concentration was determined. To exclude contamination of eluted primary S100A12 complexes with its sister molecule, S100A8/A9 (MRP8/14), identical concentrations of primary dim-, tetra-, hex-, and higher MW S100A12 were run on SDS PAGE (2×: Dimer, 4×: Tetramer, 6×: Hexamer, >6×: Hexamer-Multimer) and analyzed by Western Blot using anti-S100A12 mAb antibody (3G1D5, lanes 1-4) or anti-S1008/A9 IgG (lanes 6-9). In comparison with a S100A8/A9 positive control (lane 5), no contaminations of the individual S100A12 complexes with any S100A8/A9 could be detected.
(E) Primary, patient serum isolated S100A12 complexes as well as recombinant protein was analyzed for TNFα-induction on PMA differentiated THP-1 cells as highly sensitive reporter cell line (high TLR4/CD14 expression). All patient serum isolated complexes (hexS100A12 multimers, hexS100A12, tetraS100A12) induced strong TNFα release, which was strongly elevated above cytokine release induced by recombinant protein preparations. Primary hexS100A12 stimulation revealed the most pronounced TNFα-production. In proportion to the amount of hexS100A12 found in patient serum (as shown in C), hexS100A12 appears as the paramount stimulating complex.

FIG. 8: Detection and Isolation of S100A12 oligomers in or from patients' sera. (A) S100A12 in patients' sera was chemically cross-linked and isolated by immunoprecipitation (IP) from the sera. (B) The protein complexes from sera of patients with systemic juvenile idiopathic arthritis (sJIA), familial Mediterranean fever (FMF), pyoderma gangrenosum and acne (PAPA) were fractionated by gel filtration and the corresponding spectra plotted against a standard of recombinant S100A12-complex. (C) Cell culture supernatants from THP-1 cells were analyzed with respect to TNFα expression after stimulation with purified patient-S100A12 as well as recombinant protein.

FIG. 9: Anti-S100A12 dependent inhibition of monocyte stimulation. (A) In S100A12-complex specific binding studies, the anti-S100A12 monoclonal antibody (mAb, clone 11G7A11) raised by us preferentially recognizes hexameric S100A12. (B) In primary human monocyte stimulation experiments (pooled data from 2 independent donors) using 30 μg/ml (500 nM) LPS-free hexameric (hex) S100A12, 11G7A11 (top concentration 10 μg/ml=66 nM) effectively neutralizes TNFα-release by these cells. Half-maximal (1050) reduction of hexS100A12-induced TNFα-secretion already occurs at about 100fold lower mAb concentration compared to the hexS100A12 stimulation dose (6.6 nM 11G7A11 vs 500 nM hexS100A12).

### EXAMPLES

The human granulocyte-specific Ca²⁺-binding protein S100A12 is particularly overexpressed in autoinflammatory diseases such as juvenile idiopathic arthritis (JIA) as well as other inflammatory conditions (i.e. infections, vasculitides) and has been ascribed to the group of pro-inflammatory damage associated molecular pattern molecules (DAMPs). In order to operate as DAMP, S100A12 requires binding to cellular receptors. Although the protein was originally found to bind the receptor of advanced glycation endproducts (RAGE), it could be demonstrated that S100A12 stimulates proinflammatory cytokine production in monocytes via TLR4 instead of RAGE. DAMP:TLR4 signaling is often discussed controversially. Mechanistic insights into the protein:receptor interaction as available for HMGB1, for example, can help to explain the powerful pro-inflammatory potential of these proteins. Upon Ca²⁺ and Zn²⁺-binding S100A12 can oligomerize into di-, tetra- or hexamers. In the present invention the mechanism of the S100A12:TLR4 interaction for these individual protein complexes has been assessed. Extensive chemical cross-linking studies were performed in order to assess S100A12 oligomerization of both recombinant as well as native protein in autoinflammatory patients' sera as well as inside granulocytes. For receptor interaction studies, defined LPS-free chemically cross-linked S100A12-complexes were isolated via combined HPLC and gel filtration. TLR4-binding and signaling was tested on receptor-expressing cell lines as well as primary human cells. Cytokine expression in response to stimulation was quantified on mRNA and protein level. In the assays described herein, only combined presence of Ca²⁺ and Zn²⁺ concentrations in extracellular ionic-strength could induce S100A12 hexamer-formation. Intracellular Ca²⁺/Zn²⁺-levels could only induce oligomerization up to the tetrameric complex. Correspondingly, hexameric S100A12 could be detected in patients' serum, while this particular protein complex could not be found inside granulocytes. In vitro binding assays as well as cell stimulation experiments using chemically cross-linked HPLC-separated S100A12-oligomers revealed the S100A12-hexamer as the paramount TLR4-targeting proinflammatory active complex. Stimulation could be abrogated by interfering with TLR4-binding and, in particular, by blocking access to CD14. Accordingly, the S100A12-complex which is responsible for pro-inflammatory signaling through TLR4 could be identified by the present invention. This is of great interest for designing improved diagnostics as well as precisely targeted therapeutic approaches.

The following examples illustrate the invention. These examples should not be construed as to limit the scope of this invention. The examples are included for purposes of illustration and the present invention is limited only by the claims. It will be clear to a skilled person in the art that the invention may be practiced in other ways than as particularly described in the present description and examples. Numerous modifications and variations of the present invention are possible in light of the above teachings and, therefore, are within the scope of the appended claims.

Formation and Analysis of S100A12 Complexes

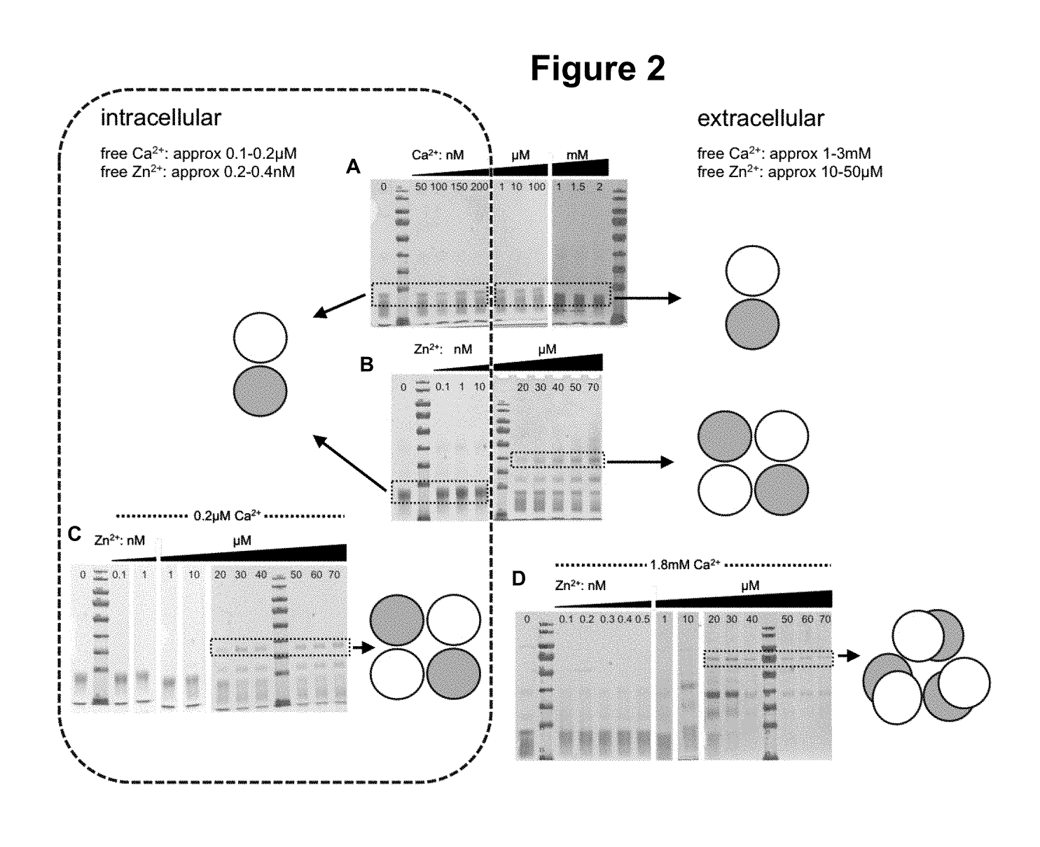

Recombinant S100A12 was expressed in E. coli and purified by anion exchange chromatography, followed by calcium-dependent hydrophobic interaction chromatography. Residual endotoxin was removed via an Endotrap column and finally quantified in EndoLISA (examples: 0.37EU LPS/ml=0.03 pg LPS/ug S100A12). After dialysis in HBS, CaCl₂ and/or ZnCl₂ in concentrations corresponding to the specified free ionic strengths were added to the purified protein in order to form complexes. The resulting S100A12 complexes were fixed by adding BS³. Excessive cross-linker was inactivated and the S100A12 complexes were separated in native form in the gel. It was found that Ca²⁺ (FIG. 2A) and Zn²⁺ (FIG. 2B) in intracellular concentration induce maximum dimers. Only extracellular Zn²⁺ concentrations (>10 μM; FIG. 2B, 2D) lead to the formation of tetramers. HexS100A12 was formed only with simultaneous presence of Ca²⁺ in extracellular ionic strength (FIG. 2D). Under physiological extracellular free Ca²⁺/Zn²⁺ on concentrations, thus, the S100A12 hexamer seems to be the primarily present complex form and is therefore of high interest as TLR4 ligand. An only Ca²⁺-induced hexameric S100A12 complex as suggested by other studies could not be reproduced in our experiments.

Isolation of Defined S100A12 Quaternary Structures

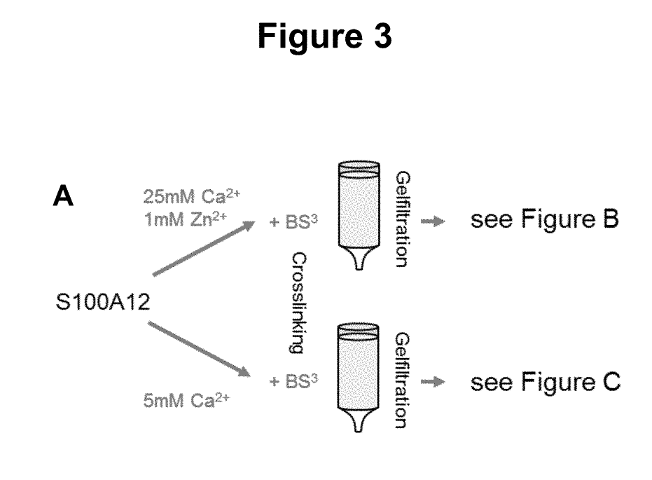

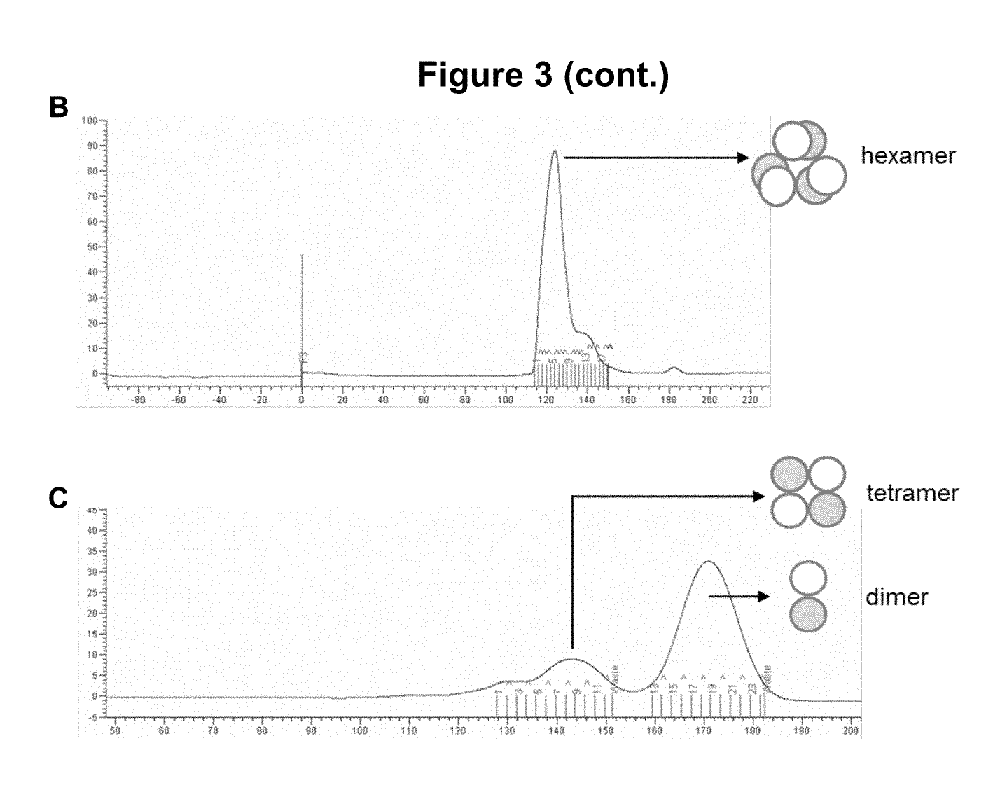

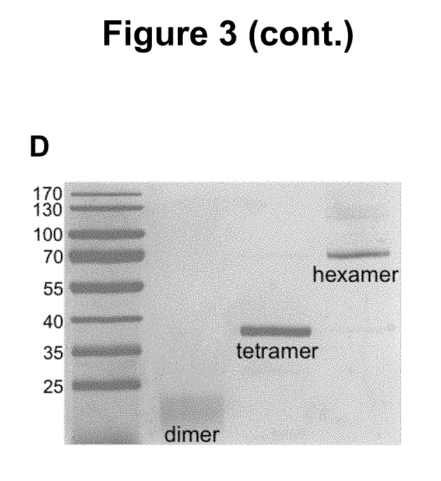

Differential TLR4 interaction studies with defined S100A12 quaternary structures require isolation of the individual complexes. Therefore, a FPLC-based gel filtration system has been established (FIG. 3). Recombinant S100A12 was prepared as described elsewhere herein and subsequently incubated with Ca²⁺/Zn²⁺ ionic strengths which leave the equilibrium of the resulting protein complexes either on the side of the smaller complexes (5 mM Ca²⁺: dimers, tetramers) or significantly shift it in the direction of hexS100A12 (25 mM Ca²⁺, 1 mM Zn²¹). The resulting quaternary structures were cross-linked with BS³ and excessive cross-linker was inactivated. The separation of the “fixed” S100A12 complexes was performed by preparative gel filtration. The protein-containing fractions were collected and analyzed for complex size in the gel. Dimer-, tetramer- and hexamer-containing eluates were each combined and concentrated using a 50 kDa Amicon filter (hexamer) or a 3 kDa Amicon filter (tetramer, dimer).

TLR4 Binding and Signaling by hexS100A12 Complexes

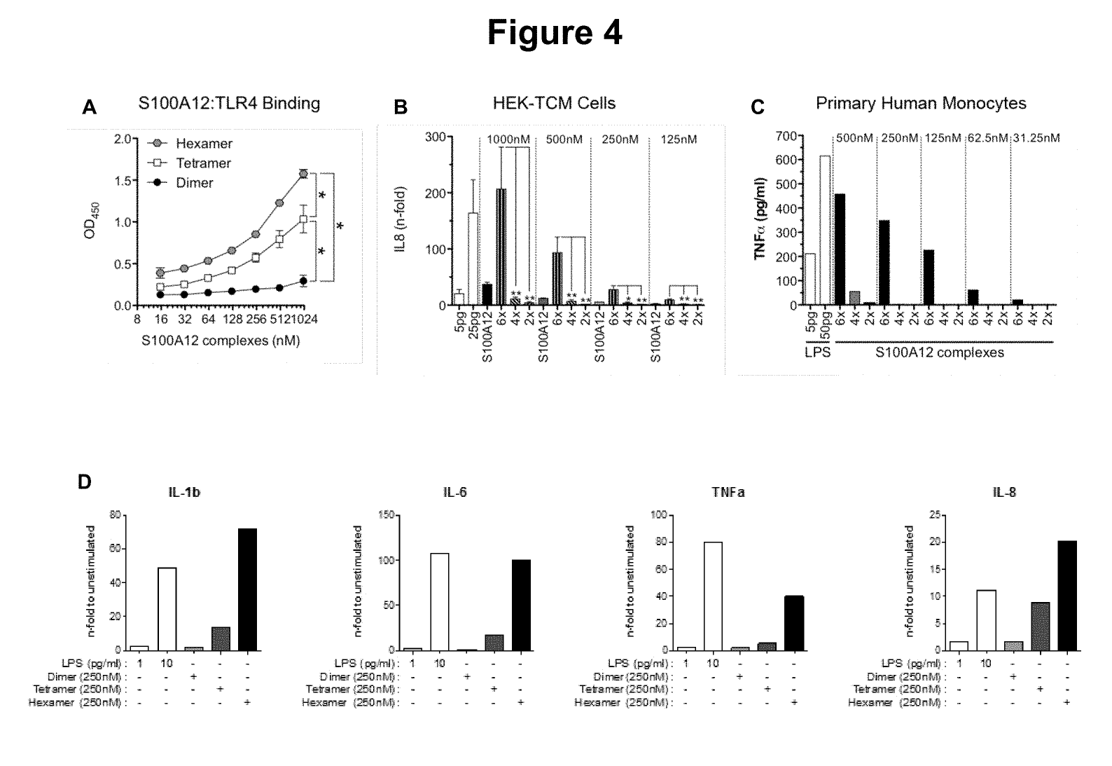

For receptor-interaction studies, defined LPS-free chemically cross-linked S100A12 complexes were isolated via combined HPLC and gel filtration. For the analysis of S100A12:TLR4 interaction a simple ELISA-based system was established. In this a recombinant human TLR4/MD2 complex (R&Dsystems, Minneapolis, USA) was immobilized on an ELISA plate and incubated with increasing concentrations of S100A12 complexes. The binding of these S100A12 complexes to the immobilized TLR4 was detected using a biotinylated anti-S100A12 antibody, followed by streptavidin-HRP. It appears from these assays, that in this system mainly hexS100A12, followed by tetS100A12 are able to bind TLR4/MD2 (FIG. 4A). A dimer binding was almost undetectable. Moreover, in particular as a result of stimulation with hexS100A12 complexes TLR4/MD2/CD14-expressing human 293 cells (HEK-TCM, InvivoGen) exhibit an upregulation of their reporter gene (IL-8) (FIG. 4B). The same applies to secretion of TNFα by hexS100a12-stimulated primary human monocytes FIG. 4C, 7E, 8C) and their IL-1b, IL-6, IL-8 and TNFα gene expression (FIG. 4D).

The Inflammatory Stimulation by hexS100A12 Complexes is Carried Out in Strict Dependence on CD14

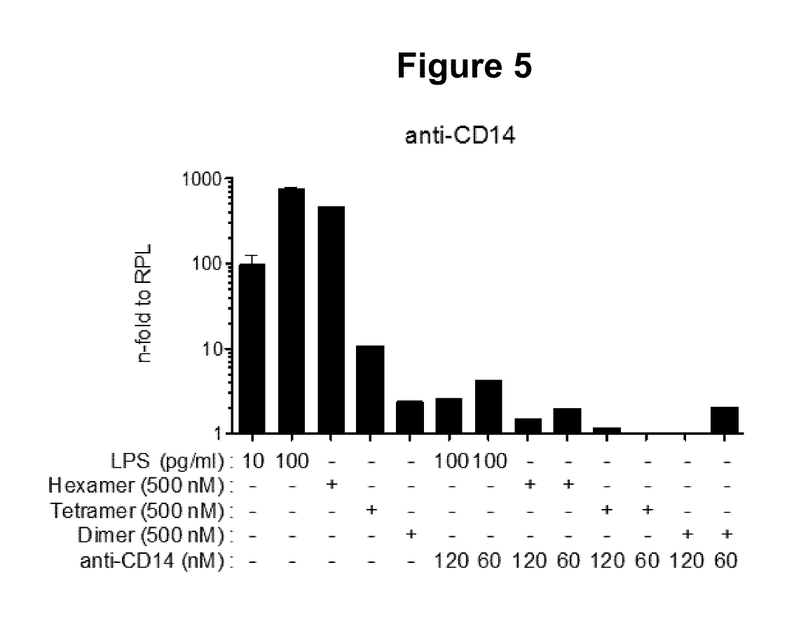

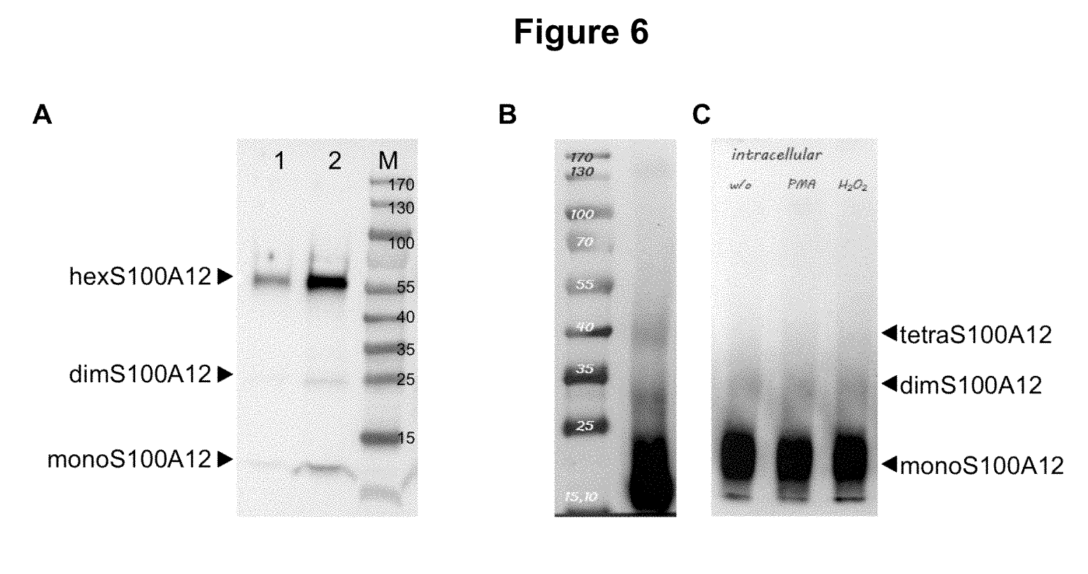

TLR4-binding and signaling was tested on TLR4/MD2 receptor-expressing cell lines as well as primary human cells. Cytokine expression in response to stimulation was quantified on mRNA and protein level. The formation of a hetero-tetrameric TLR4/MD2 complex (2×TLR4/MD2) and CD14 are responsible for an optimal signal induction. In inhibition assays on TLR4/MD2/CD14-expressing human 293 cells (HEK-TCM, InvivoGen) it was shown that CD14 is critically relevant for TLR4-dependent signaling by hexameric S100A12 (FIG. 5).

Isolation and Testing of Hexameric S100A12 Complexes from Patient Serum

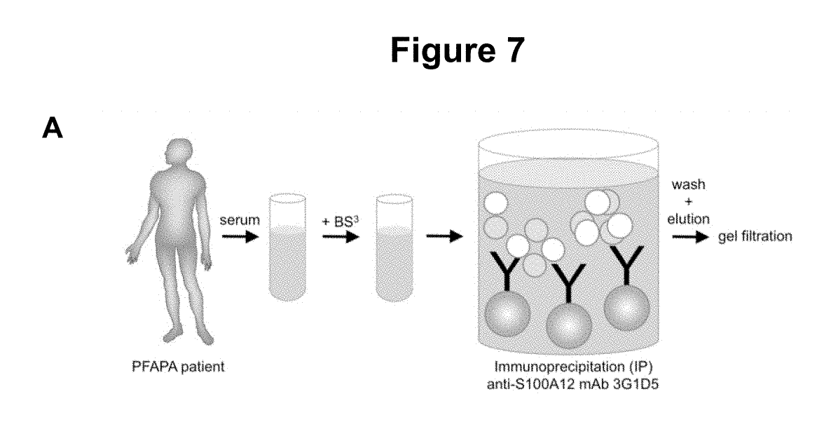

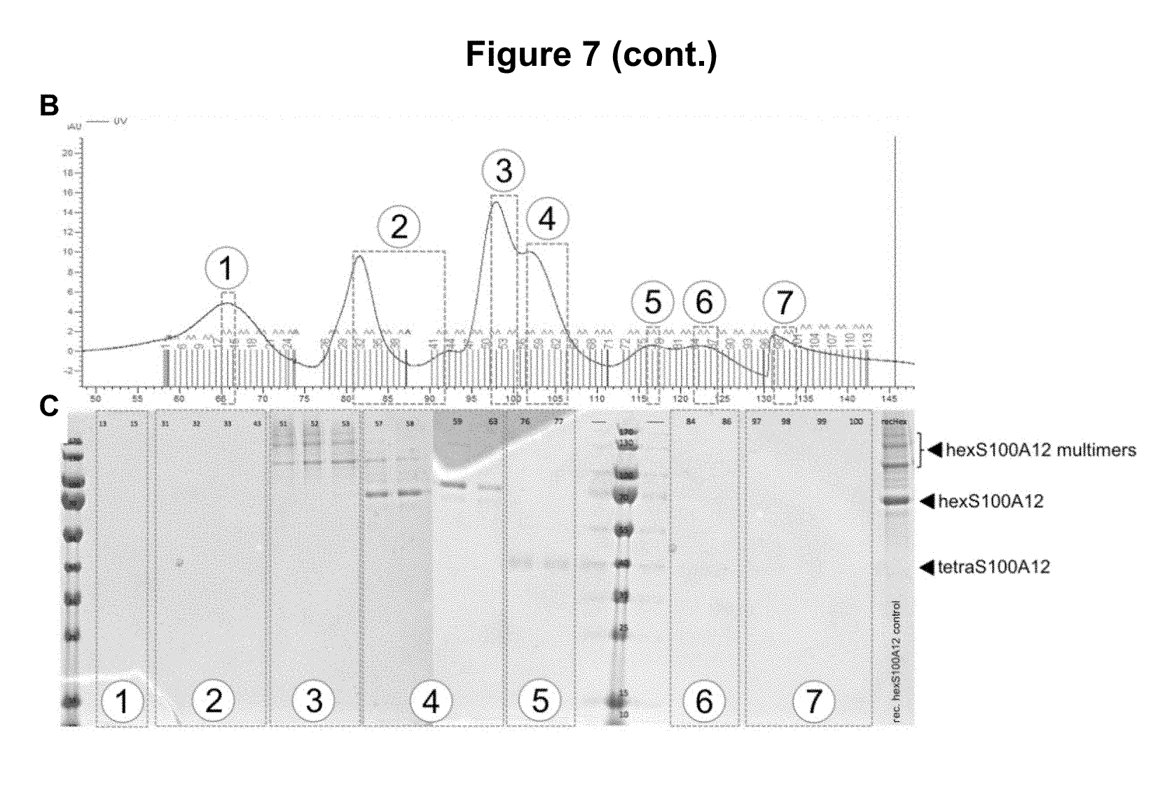

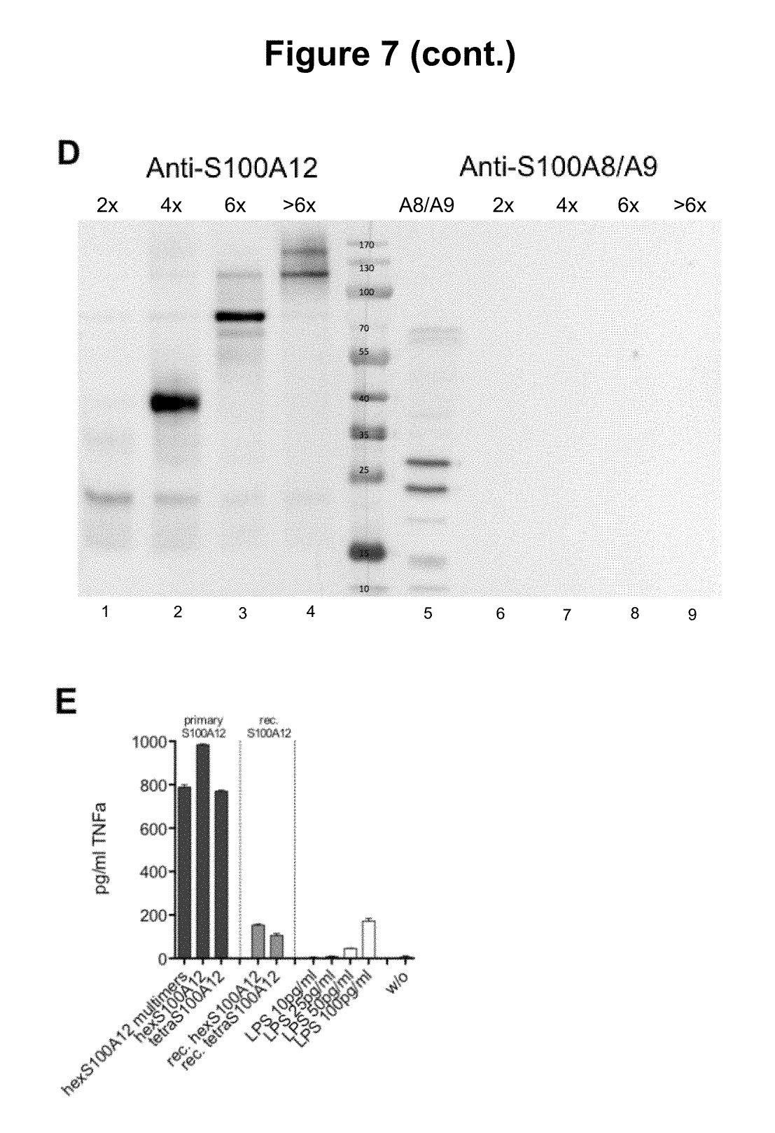

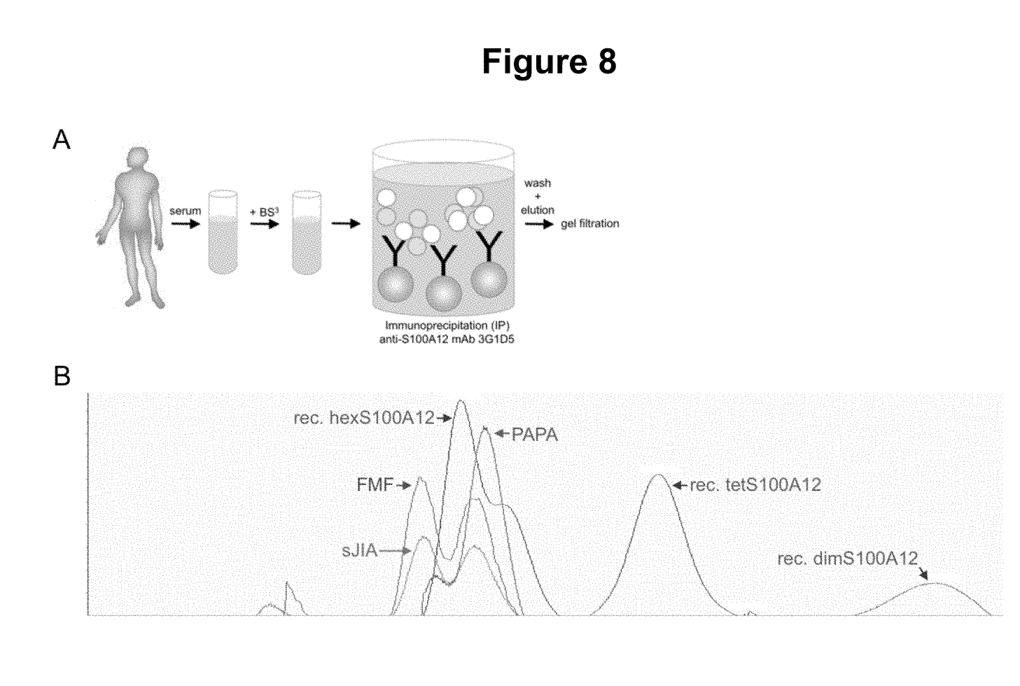

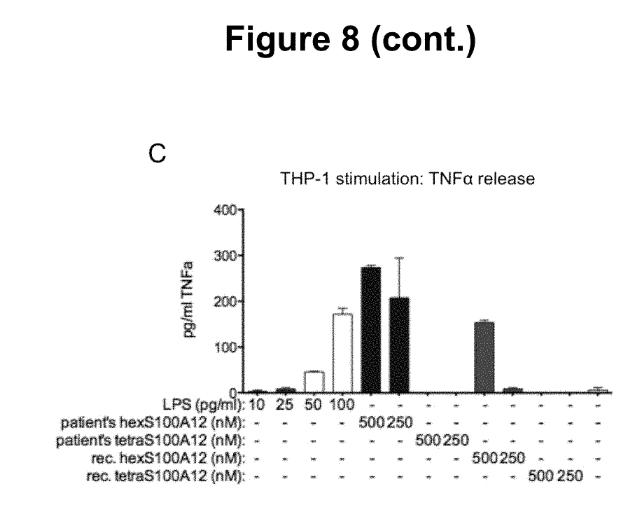

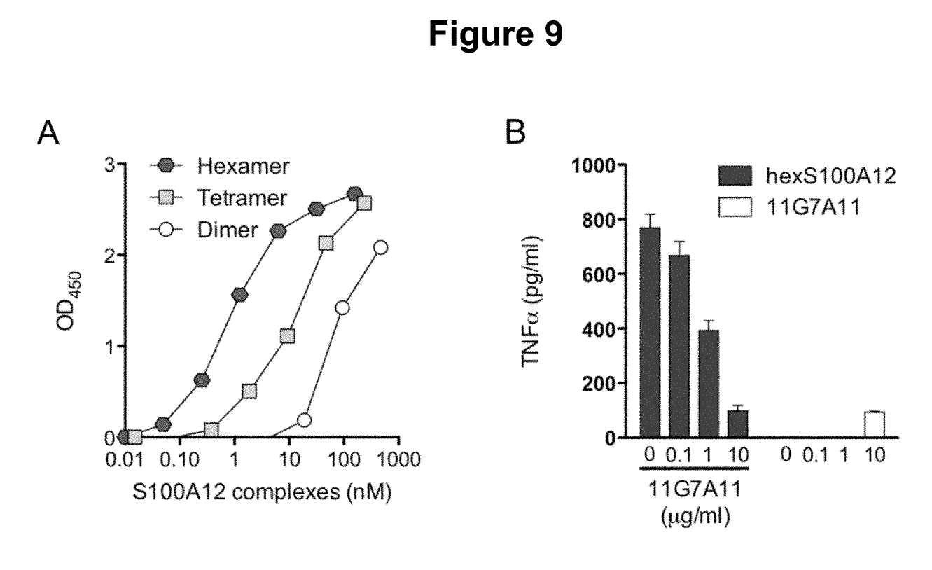

Serum of an autoinflammatory diseased patient (PFAPA: Periodic fever, aphthous stomatitis, pharyngitis, adenitis syndrome, juvenile idiopathic arthritis (sJIA), familial Mediterranean fever (FMF), pyoderma gangrenosum and acne (PAPA)) was treated with BS³ and S100A12 was precipitated using anti-S100A12 monoclonal antibody (3G1D5, produced in-house) covalently linked to magnetic beads (FIG. 7, 8). Beads were washed thoroughly to remove unspecifically bound proteins. mAb-bound proteins were eluted by pH-shift. Subsequently, pH-neutralized eluted protein was separated by gel filtration.
Analysis of protein fractions isolated from the dominant protein peaks eluting from gel filtration on SDS PAGE revealed hexS100A12 as dominant band appearing in Coomassie stain. This is followed by bands resembling multimers of hexS100A12 and a weak tetraS100A12 band. DimS100A12 cannot be detected in serum.
Patient serum isolated S100A12 complexes as well as recombinant protein were analyzed for TNFα-induction on PMA differentiated THP-1 cells as highly sensitive reporter cell line (high TLR4/CD14 expression). All patient serum isolated complexes (hexS100A12 multimers, hexS100A12, tetraS100A12) induced strong TNFα release. This was highly elevated above cytokine release induced by recombinant protein preparations. Primary hexS100A12 stimulation revealed the most pronounced TNFα-production. In proportion to the amount of hexS100A12 found in patient serum, hexS100A12 appears as the paramount pro-inflammatory complex (FIG. 7, 8).

Generation of Antibodies Against Hexameric S100A12 Complexes

Wistar rats are immunized repeatedly with BS³-crosslinked S100A12, generated as described in 78 and 79. Animals are bled and sera are checked for hexS100A12 antibody responses. Already at this stage, sera are counter-screened for recognition of tetra- and/or dimeric S100A12 (generated as described in 78 and 79) to identify animals with a preferential anti-hexS100A12 IgG-response. Splenic B cell blasts of selected animals are fused with myeloma cells. Parental supernatants are analyzed for anti-hexS100A12 IgG and counter-screened for recognition of tetra- or dimeric S100A12. Only parental clones with an exclusive hexS100A12-specific IgG-response are subjected to subcloning. After three rounds of subcloning, monoclonal antibodies are again checked for their exclusive hexS100A12 specificity. Large scale expression of successful clones is performed in roller bottle cultures.

1. An in vitro method of diagnosing the risk of occurrence or the presence of a S100A12:TLR4/MD2/CD14-mediated inflammatory disorder in a subject, comprising:
(a) determining the amount of hexameric S100A12 complex in a biological sample obtained from said subject, and
(b) comparing the amount of hexameric S100A12 complex determined in (a) with a control sample.

2. The method according to claim 1, wherein an increased amount of hexameric S100A12 complex as compared to the control sample is indicative of an elevated risk of occurrence or the presence of an inflammatory disorder.

3. The method according to claim 1, wherein said S100A12:TLR4/MD2/CD14-mediated inflammatory disorder is characterized by an increased amount of extracellular hexameric S100A12 complex in said subject.

4. The method according to claim 1, wherein said extracellular hexameric S100A12 complex is a Ca²⁺/Zn²⁺-dependent hexameric S100A12 complex.

5. (canceled)

6. The method according to claim 1, wherein said S100A12:TLR4/MD2/CD14-mediated inflammatory disorder is further associated with an increased expression and/or accumulation of TNFα, IL-8, IL-1b, and IL-6.

7. The method according to claim 1, wherein said S100A12:TLR4/MD2/CD14-mediated inflammatory disorder is
(a) an acute or chronic auto-inflammatory disease selected from the group consisting of inflammatory bowel diseases, juvenile idiopathic arthritis (JIA), systemic juvenile idiopathic arthritis (sJIA), rheumatoid and psoriatic arthritis, seronegative arthritis
(b) a local or systemic infection,
(c) vasculitides,
(d) cancer,
(e) a kidney disease or malfunction,
(f) lung injury or pulmonary disease,
(g) allergy,
(h) a cardiovascular disease,
(i) familial Mediterranean fever (FMF), or
(j) pyoderma gangrenosum and acne (PAPA).

8. The method according to claim 1, wherein said biological sample is a serum sample, a plasma sample, an urine sample, a feces sample, a saliva sample, a tear fluid sample, or a tissue extract sample.

9-11. (canceled)

12. The method according to claim 1, wherein determining the amount of hexameric S100A12 complex in said biological sample comprises the use of an immunoglobulin having a binding specificity to hexameric S100A12 complex but no binding specificity to tetrameric or dimeric S100A12 complex or monomeric S100A12.

13. (canceled)

14. (canceled)

15. An immunoglobulin having a binding specificity to hexameric S100A12 complex but no binding specificity to tetrameric or dimeric S100A12 complex or monomeric S100A12.

16. (canceled)

17. (canceled)

18. The immunoglobulin according to claim 15, wherein said hexameric S100A12 complex is a Ca²⁺/Zn²⁺-dependent hexameric S100A12 complex.

19. The immunoglobulin according to claim 15, wherein the immunoglobulin specifically inhibits the interaction of hexameric S100A12 complex to the TLR4/MD2/CD14 complex.

20. (canceled)

21. The immunoglobulin according to claim 15, wherein the immunoglobulin significantly decreases the expression of TNFα, IL-8, IL-1b, and IL-6.

22. The immunoglobulin according to claim 15, wherein the immunoglobulin is a monoclonal immunoglobulin or a fragment thereof.

23-43. (canceled)

44. A method for the treatment of a subject suffering from a S100A12:TLR4/MD2/CD14-mediated inflammatory disorder, the method comprising administering a therapeutically effective amount of a compound having a binding specificity to hexameric S100A12 complex but no binding specificity to tetrameric or dimeric S100A12 complex or monomeric S100A12 to a subject in need thereof.

45. The method according to claim 44, wherein said compound specifically inhibits the interaction of hexameric S100A12 complex to the TLR4/MD2/CD14 complex.

46. (canceled)

47. The method according to claim 44, wherein said compound significantly decreases the expression of TNFα, IL-8, IL-1b, and IL-6.

48. The method according to claim 44, wherein said compound is an immunoglobulin having a binding specificity to hexameric S100A12 complex but no binding specificity to tetrameric or dimeric S100A12 complex or monomeric S100A12.

49. The method according to claim 44, wherein said S100A12:TLR4/MD2/CD14-mediated inflammatory disorder is characterized by an increased amount of extracellular hexameric S100A12 complex in said subject.

50. (canceled)

51. (canceled)

52. The method according to claim 44, wherein said S100A12:TLR4/MD2/CD14-mediated inflammatory disorder is further associated with an increased expression and/or accumulation of TNFα, IL-8, IL-1b, and IL-6.

53. The method according to claim 44, wherein said S100A12:TLR4/MD2/CD14-mediated inflammatory disorder is
(a) an acute or chronic auto-inflammatory disease selected from the group consisting of inflammatory bowel diseases, juvenile idiopathic arthritis (JIA), systemic juvenile idiopathic arthritis (sJIA), rheumatoid and psoriatic arthritis, seronegative arthritis
(b) a local or systemic infection,
(c) vasculitides,
(d) cancer,
(e) a kidney disease or malfunction,
(f) lung injury or pulmonary disease,
(g) allergy,
(h) a cardiovascular disease,
(i) familial Mediterranean fever (FMF), or
(j) pyoderma gangrenosum and acne (PAPA).

54-56. (canceled)
